(19) United States
(12) Patent Application Publication
(10) Pub. No.: US 2016/0099419 A1
(43) Pub. Date: Apr. 7, 2016

(54) ORGANIC ELECTROLUMINESCENT MATERIAL AND ORGANIC ELECTROLUMINESCENT DEVICE INCLUDING THE SAME

(71) Applicant: Samsung Display Co., Ltd., Yongin-si (KR)

(72) Inventor: Naoya SAKAMOTO, Yokohama (JP)

(21) Appl. No.: 14/859,218

(22) Filed: Sep. 18, 2015

(30) Foreign Application Priority Data
Oct. 2, 2014 (JP) 2014-204209

(51) Int. Cl. H01L 51/00; C07D 405/14; C07D 409/14; C07D 403/04; C07D 403/14; C07D 405/04; C07D 401/14; C07D 245/04; C07F 7/08

(52) U.S. Cl. CPC H01L 51/0061; C07D 245/04; H01L 51/006; C07D 405/14; C07D 409/14; C07F 7/0816; H01L 51/0094; C07D 403/14; C07F 7/0812; C07D 405/04; C07D 401/14; C07D 403/04; H01L 51/5016

(57) ABSTRACT

A material for an organic electroluminescent device according to embodiments of the present disclosure is represented by the following Formula (1). The material for an organic electroluminescent device may have high emission efficiency and the organic electroluminescent device including the material may have improved characteristics.

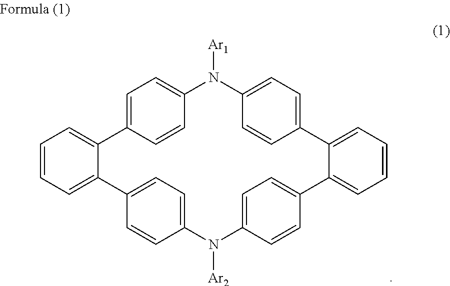

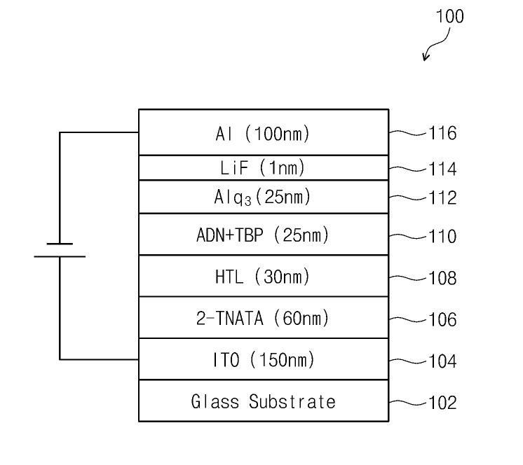

### CROSS-REFERENCE TO RELATED APPLICATION

This U.S. non-provisional patent application claims priority to and the benefit of Japanese Patent Application No. 2014-204209, filed on Oct. 2, 2014, the entire content of which is hereby incorporated by reference.

### BACKGROUND

1. Field

One or more aspects of embodiments of the present disclosure relate to an organic electroluminescent material and an organic electroluminescent device including the same. More particularly, one or more aspects of embodiments of the present disclosure relate to an organic electroluminescent material exhibiting high emission efficiency in a blue emission region, and an organic electroluminescent device including the same.

2. Description of the Related Art

Recently, the development of an organic electroluminescent display (organic EL display) is being actively conducted. The organic EL display, unlike a liquid crystal display or the like, is a self-luminescent display capable of displaying images by recombining holes and electrons from an anode and a cathode in an emission layer and by emitting light from a luminescent material including an organic compound in the emission layer.

For example, an organic electroluminescent device (organic EL device) may include an anode, a hole transport layer positioned on the anode, an emission layer positioned on the hole transport layer, an electron transport layer positioned on the emission layer and a cathode positioned on the electron transport layer. Holes are injected from the anode and move via the hole transport layer into the emission layer. Electrons are injected from the cathode and move via the electron transport layer into the emission layer. Through the recombination of the holes and electrons injected into the emission layer, excitons are generated in the emission layer. The organic EL device emits lights generated by the radiation deactivation of these excitons. The configuration of the organic EL device is not limited thereto, and may include various suitable modifications.

For the improved application of the organic EL device in a display, the increase of the emission efficiency of the organic EL device may be required. Particularly, a blue emission region, as compared to a red emission region and a green emission region of the organic EL device, may exhibit insufficient emission efficiency. To realize high emission efficiency of the organic EL device, the normalization and stabilization of a hole transport layer, for example, has been examined. For example, an aromatic amine compound has been utilized as a hole transport material in a hole transport layer. For example, a para-phenylenediamine derivative has been utilized as a hole transport material. However, the para-phenylenediamine derivative has a high degree of the highest occupied molecular orbital (HOMO) level, and the organic EL device using the para-phenylenediamine derivative may not obtain sufficient emission efficiency. A tertiary amine derivative having a ring shape (e.g., ring structure) has also been utilized as the hole transport material. However, in such amine derivative, a conjugation structure may be formed between amines, and thus the HOMO level may be increased and sufficient emission efficiency may not be obtained.

Accordingly, there is a need for an organic EL device having improved emission efficiency. Particularly, since the emission efficiency of the organic EL device is relatively low in the blue emission region, as compared to the red emission region and the green emission region, the improvement of the emission efficiency is required in the blue emission region. To increase the emission efficiency of the organic EL device further, the development of a novel material is necessary.

### SUMMARY

One or more aspects of embodiments of the present disclosure provide a material for an organic EL device having high emission efficiency and an organic EL device including the same.

In some embodiments, the present disclosure provides a material for an organic EL device having high emission efficiency in a blue emission region, the material being used in an emission layer or at least one layer selected from the stacked layers positioned between the emission layer and an anode, and an organic EL device including the same.

In one or more embodiments of the present disclosure, a material for an organic EL device is represented by Formula (1):

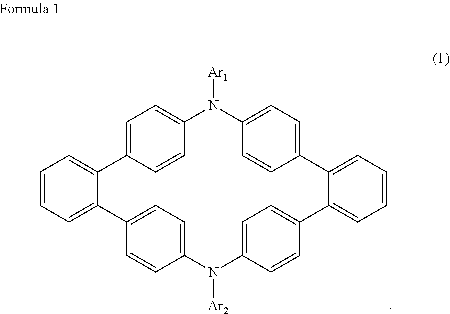

In the above Formula (1), Ar₁ and Ar₂ may be each independently a substituted or unsubstituted aryl group or a substituted or unsubstituted heteroaryl group.

In the material for an organic EL device according to an embodiment of the present disclosure, since tertiary amines are connected via o-terphenyl groups (ortho-terphenyl groups), conjugation between amines may not be formed, and an appropriate HOMO level may be realized. In addition, since the material has a ring-shaped structure (e.g., ring structure), heat resistance may be improved, and the decomposition of the material itself may be restrained or reduced. In addition, since an energy gap is relatively large, high emission efficiency may be realized by using the material as a phosphorescent host material or a hole transport material in a hole transport layer, when the hole transport layer is adjacent to the phosphorescent emission layer. For example, improved properties of the organic EL device may be obtained in a blue region.

In other embodiments of the present disclosure, organic EL devices may include the material for an organic EL device in one layer selected from the stacked layers positioned between an emission layer and an anode of the organic EL device.

According to embodiments of the present disclosure, by using a diamine derivative having an o-terphenyl skeleton (e.g., including o-terphenyl groups) and a ring shape (e.g., ring structure) in a layer selected from the stacked layers between an emission layer and an anode in the organic EL device, an energy gap may be increased, and high emission efficiency may be realized. For example, improved properties of the organic EL device may be obtained in a blue region.

In some embodiments of the present disclosure, organic EL devices may include the material for an organic EL device in an emission layer.

According to embodiments of the present disclosure, by using a diamine derivative having an o-terphenyl skeleton (e.g., including o-terphenyl groups) and a ring shape (e.g., ring structure) in an emission layer in the organic EL device, an energy gap may be increased, and high emission efficiency may be realized. For example, improved properties may be obtained in a blue region.

According to embodiments of the present disclosure, a material for an organic EL device having high emission efficiency and an organic EL device including the same may be provided. For example, a material for an organic EL device having high emission efficiency in a blue emission region and used in an emission layer or a layer selected from the stacked layers positioned between the emission layer and an anode, and an organic EL device including the same, may be provided. Since the material according to embodiments of the present disclosure includes tertiary amines that are connected via o-terphenyl groups, conjugation between amines may not be formed, and an appropriate HOMO level may be realized. In addition, since the material for an organic EL device has a ring-shaped structure (e.g., ring structure), heat resistance may be improved, and the decomposition of the material itself may be restrained or reduced. In addition, since an energy gap of the material for an organic EL device is relatively large, an organic EL device with high emission efficiency may be realized by using the material as a phosphorescent host material or a hole transport material in a hole transport layer, when the hole transport layer is adjacent to the phosphorescent emission layer.

### BRIEF DESCRIPTION OF THE DRAWING

The accompanying drawing is included to provide a further understanding of the present disclosure, and is incorporated in and constitutes a part of this specification. The drawing illustrates example embodiments of the present disclosure and, together with the description, serves to explain principles of the present disclosure. The drawing is a schematic view of an organic EL device 100 according to an embodiment of the present disclosure.

### DETAILED DESCRIPTION

According to one or more embodiments of the present disclosure, high emission efficiency of an organic EL device may be obtained by employing ortho-terphenyl groups (herein, o-terphenyl).

Hereinafter, the material for an organic EL device and an organic EL device including the same according to embodiments of the present disclosure will be explained referring to the drawing. The material for an organic EL device and an organic EL device including the same according to embodiments of the present disclosure may, however, be embodied in different forms and should not be constituted as limited to the embodiments set forth herein. In the drawing and in the present specification, the same reference numeral may be designated for the same parts (elements) or parts (elements) having the same function, and repeated explanations thereof will not be provided.

The material for an organic EL device according to embodiments of the present disclosure may include a diamine derivative having a ring shape (e.g., ring structure) obtained by connecting two tertiary amines via o-terphenyl, and may be represented by Formula (1):

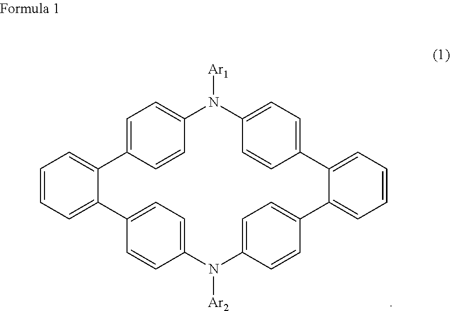

In Formula (1), Ar₁ and Ar₂ may be each independently a substituted or unsubstituted aryl group or a substituted or unsubstituted heteroaryl group, and Ar₁ and Ar₂ may be the same as or different from each other.

Here, as the substituted or unsubstituted aryl group used in, for example, Ar₁ and Ar₂, a substituted or unsubstituted aryl group having 6 to 30 carbon atoms for forming a ring may be used, and non-limiting examples thereof may include a phenyl group, a naphthyl group, an anthracenyl group, a phenanthryl group, a biphenyl group, a terphenyl group, a quaterphenyl group, a quniqphenyl group, a sexyphenyl group, a fluorenyl group, a triphenylene group, a biphenylene group, a pyrenyl group, a benzofluoranthenyl group, a glyceryl group, and the like.

As the substituted or unsubstituted heteroaryl group used in, for example, Ar₁ and Ar₂, a heteroaryl group having 6 to 30 carbon atoms for forming a ring may be used, and non-limiting examples thereof may include a benzothiazolyl group, a thiophenyl group, a thienothiophenyl group, a benzothiophenyl group, a benzofuryl group, a dibenzothiophenyl group, a dibenzofuryl group, a N-arylcarbazolyl group, a N-heteroarylcarbazolyl group, a N-alkylcarbazolyl group, a phenoxazyl group, a phenothiazyl group, a pyridyl group, a pyrimidyl group, a triazinyl group, a quinolinyl group, a quinoxalyl group, and the like. As used herein, the statement “atoms for forming a ring” may refer to “ring-forming atoms.”

In the material for an organic EL device according to embodiments of the present disclosure, since two tertiary amines are connected via o-terphenyl, conjugation between amines may not be formed, and an appropriate highest occupied molecular orbital (HOMO) level may be realized. In addition, since the material for an organic EL device according to embodiments of the present disclosure has a ring shaped structure (e.g., ring structure), heat resistance may be improved, and the decomposition of the material itself may be restrained or reduced. In addition, since an energy gap of the material for an organic EL device according to embodiments of the present disclosure is relatively large, an organic EL device with high emission efficiency may be realized by using the material as a phosphorescent host material or a hole transport material in a hole transport layer, when the hole transport layer is adjacent to the phosphorescent emission layer.

In some embodiments, the material for an organic EL device according to embodiments of the present disclosure may include at least one selected from Compounds 1 to 5.

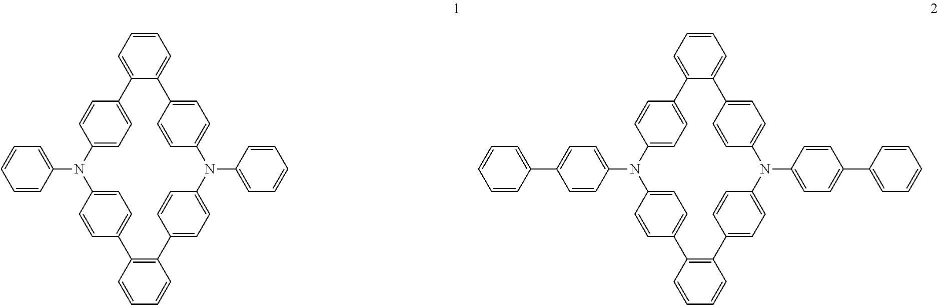 [SMILES: c1ccc(N2c3ccc(cc3)-c3ccccc3-c3ccc(cc3)N(c3ccccc3)c3ccc(cc3)-c3ccccc3-c3ccc2cc3)cc1 ; c1ccc(-c2ccc(N3c4ccc(cc4)-c4ccccc4-c4ccc(cc4)N(c4ccc(-c5ccccc5)cc4)c4ccc(cc4)-c4ccccc4-c4ccc3cc4)cc2)cc1]

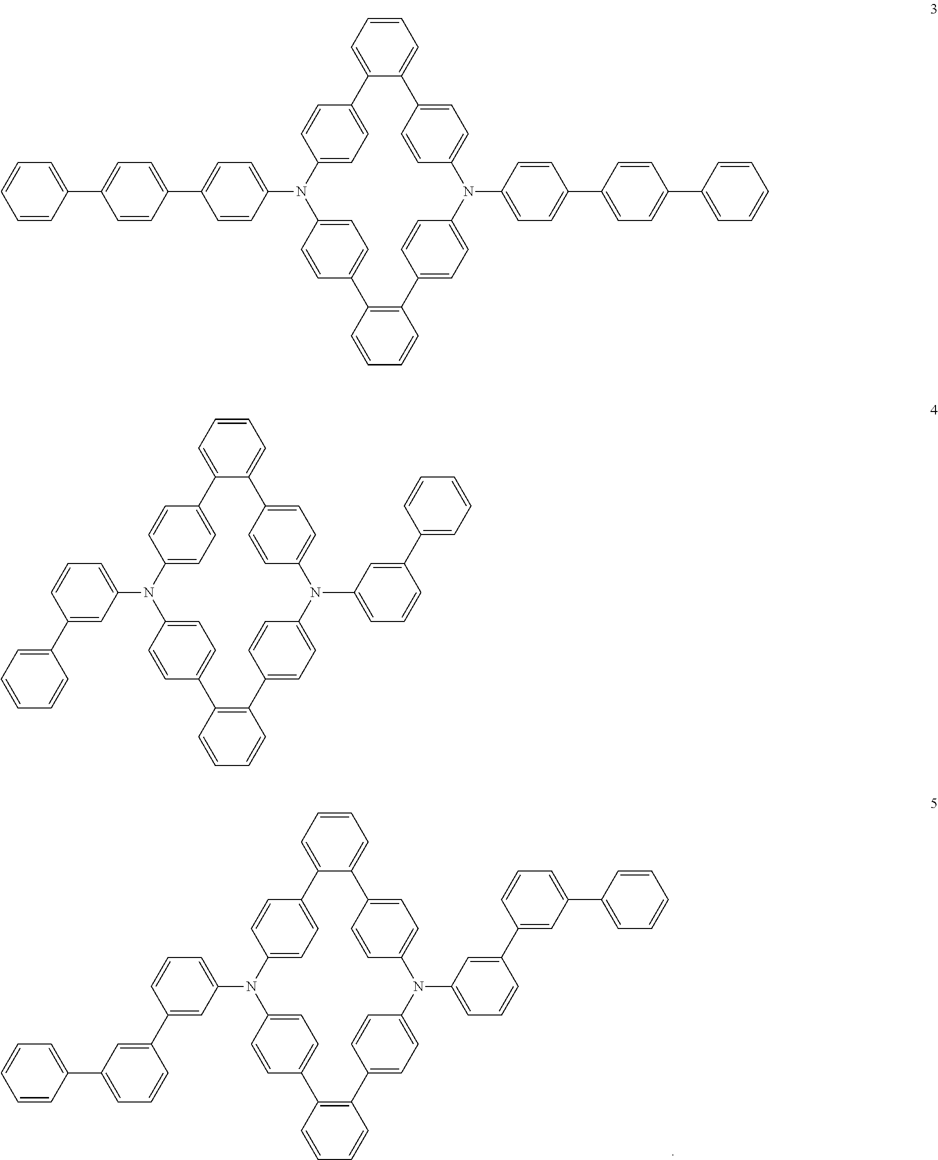 [SMILES: c1ccc(-c2ccc(-c3ccc(N4c5ccc(cc5)-c5ccccc5-c5ccc(cc5)N(c5ccc(-c6ccc(-c7ccccc7)cc6)cc5)c5ccc(cc5)-c5ccccc5-c5ccc4cc5)cc3)cc2)cc1 ; c1ccc(-c2cccc(N3c4ccc(cc4)-c4ccccc4-c4ccc(cc4)N(c4cccc(-c5ccccc5)c4)c4ccc(cc4)-c4ccccc4-c4ccc3cc4)c2)cc1 ; c1ccc(-c2cccc(-c3cccc(N4c5ccc(cc5)-c5ccccc5-c5ccc(cc5)N(c5cccc(-c6cccc(-c7ccccc7)c6)c5)c5ccc(cc5)-c5ccccc5-c5ccc4cc5)c3)c2)cc1]

In some embodiments, the material for an organic EL device according to embodiments of the present disclosure may include at least one selected from Compounds 6 to 11.

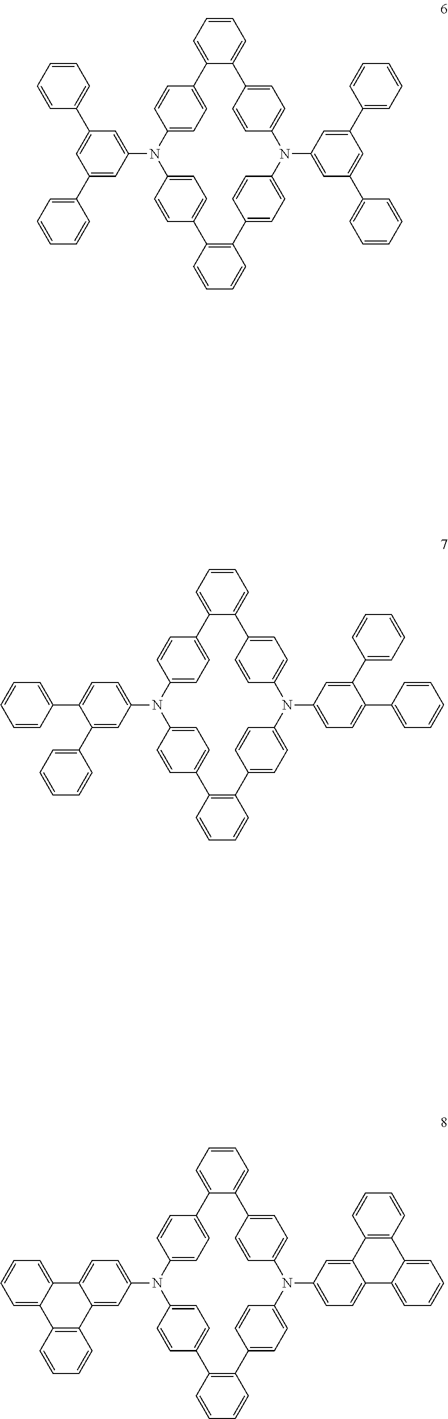 [SMILES: c1ccc(-c2cc(-c3ccccc3)cc(N3c4ccc(cc4)-c4ccccc4-c4ccc(cc4)N(c4cc(-c5ccccc5)cc(-c5ccccc5)c4)c4ccc(cc4)-c4ccccc4-c4ccc3cc4)c2)cc1 ; c1ccc(-c2ccc(N3c4ccc(cc4)-c4ccccc4-c4ccc(cc4)N(c4ccc(-c5ccccc5)c(-c5ccccc5)c4)c4ccc(cc4)-c4ccccc4-c4ccc3cc4)cc2-c2ccccc2)cc1 ; c1ccc2c(c1)-c1ccc(cc1)N(c1ccc3c4ccccc4c4ccccc4c3c1)c1ccc(cc1)-c1ccccc1-c1ccc(cc1)N(c1ccc3c4ccccc4c4ccccc4c3c1)c1ccc-2cc1]

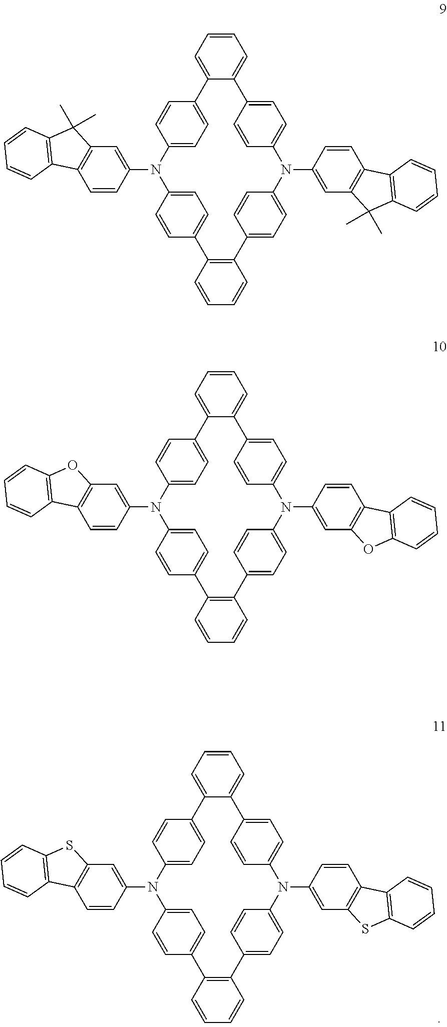 [SMILES: CC1(C)c2ccccc2-c2ccc(N3c4ccc(cc4)-c4ccccc4-c4ccc(cc4)N(c4ccc5c(c4)C(C)(C)c4ccccc4-5)c4ccc(cc4)-c4ccccc4-c4ccc3cc4)cc21 ; c1ccc2c(c1)-c1ccc(cc1)N(c1ccc3c(c1)oc1ccccc13)c1ccc(cc1)-c1ccccc1-c1ccc(cc1)N(c1ccc3c(c1)oc1ccccc13)c1ccc-2cc1 ; c1ccc2c(c1)-c1ccc(cc1)N(c1ccc3c(c1)sc1ccccc13)c1ccc(cc1)-c1ccccc1-c1ccc(cc1)N(c1ccc3c(c1)sc1ccccc13)c1ccc-2cc1]

In some embodiments, the material for an organic EL device according to embodiments of the present disclosure may include at least one selected from Compounds 12 to 17.

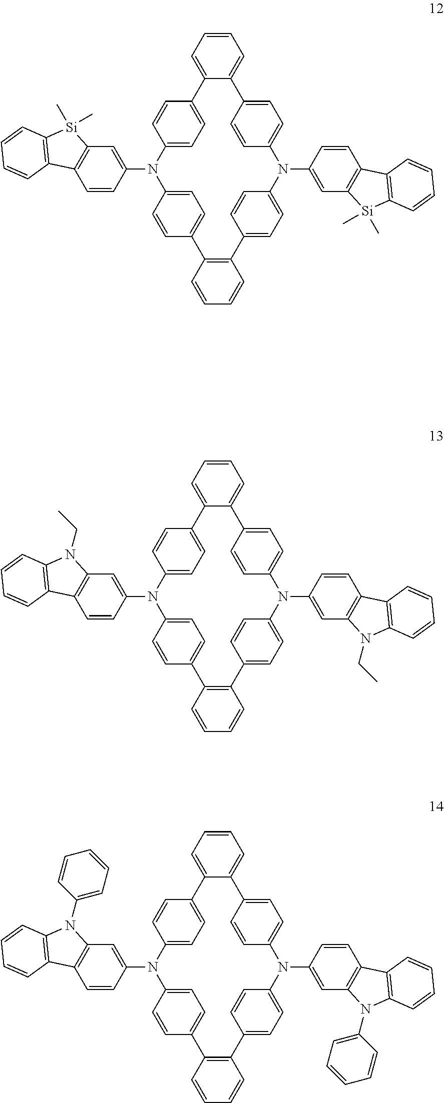 [SMILES: C[Si]1(C)c2ccccc2-c2ccc(N3c4ccc(cc4)-c4ccccc4-c4ccc(cc4)N(c4ccc5c(c4)[Si](C)(C)c4ccccc4-5)c4ccc(cc4)-c4ccccc4-c4ccc3cc4)cc21 ; CCn1c2ccccc2c2ccc(N3c4ccc(cc4)-c4ccccc4-c4ccc(cc4)N(c4ccc5c6ccccc6n(CC)c5c4)c4ccc(cc4)-c4ccccc4-c4ccc3cc4)cc21 ; c1ccc(-n2c3ccccc3c3ccc(N4c5ccc(cc5)-c5ccccc5-c5ccc(cc5)N(c5ccc6c7ccccc7n(-c7ccccc7)c6c5)c5ccc(cc5)-c5ccccc5-c5ccc4cc5)cc32)cc1]

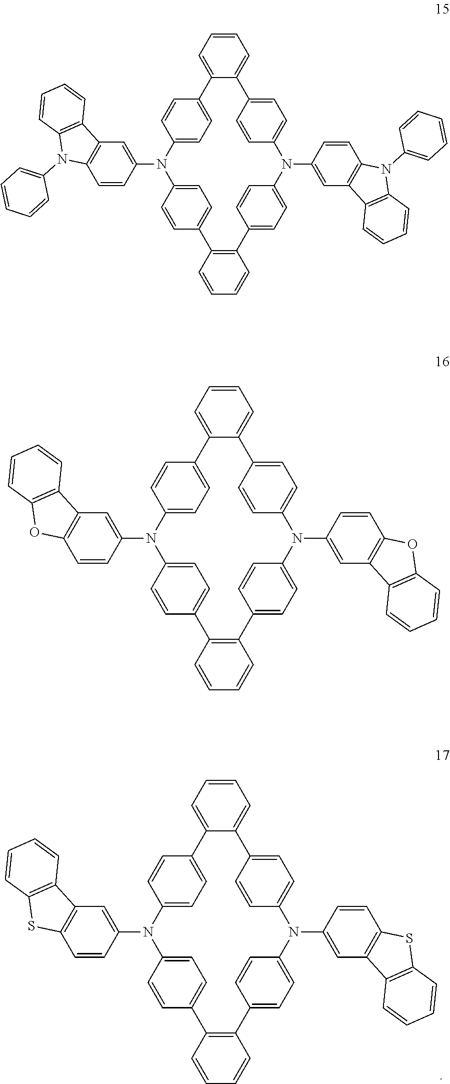 [SMILES: c1ccc(-n2c3ccccc3c3cc(N4c5ccc(cc5)-c5ccccc5-c5ccc(cc5)N(c5ccc6c(c5)c5ccccc5n6-c5ccccc5)c5ccc(cc5)-c5ccccc5-c5ccc4cc5)ccc32)cc1 ; c1ccc2c(c1)-c1ccc(cc1)N(c1ccc3oc4ccccc4c3c1)c1ccc(cc1)-c1ccccc1-c1ccc(cc1)N(c1ccc3oc4ccccc4c3c1)c1ccc-2cc1 ; c1ccc2c(c1)-c1ccc(cc1)N(c1ccc3sc4ccccc4c3c1)c1ccc(cc1)-c1ccccc1-c1ccc(cc1)N(c1ccc3sc4ccccc4c3c1)c1ccc-2cc1]

In some embodiments, the material for an organic EL device according to embodiments of the present disclosure may include at least one selected from Compounds 18 to 23.

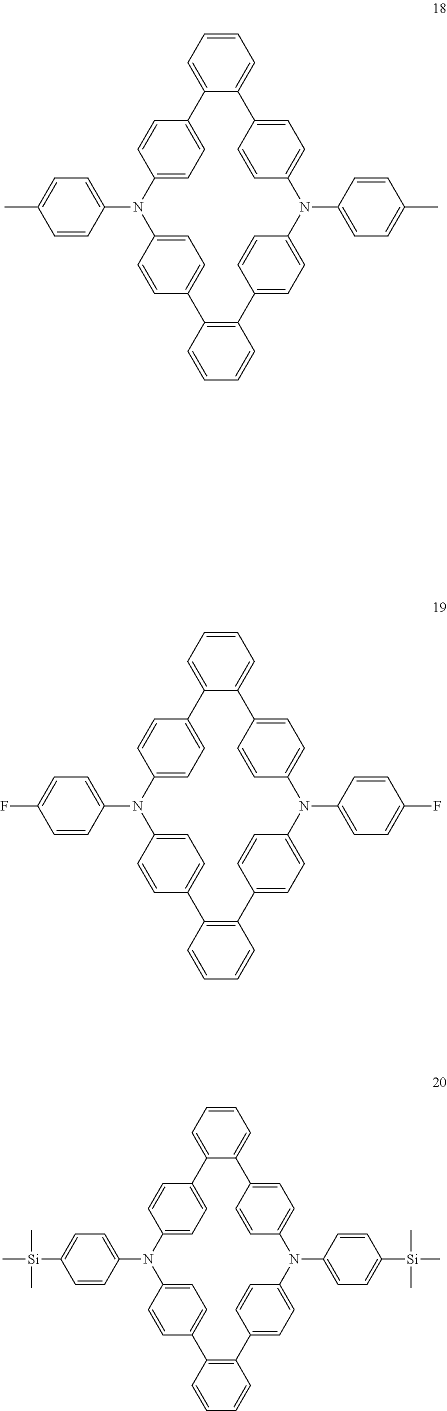

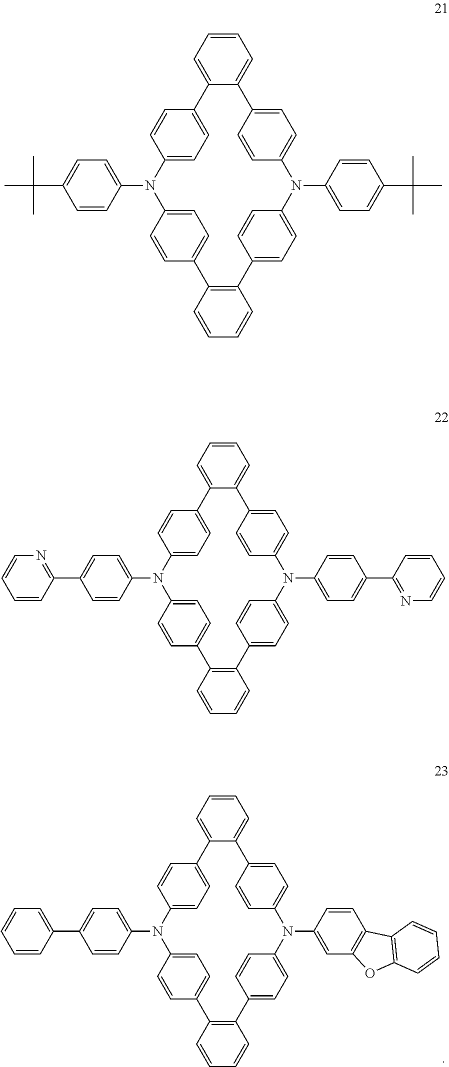 [SMILES: CC(C)(C)c1ccc(N2c3ccc(cc3)-c3ccccc3-c3ccc(cc3)N(c3ccc(C(C)(C)C)cc3)c3ccc(cc3)-c3ccccc3-c3ccc2cc3)cc1 ; c1ccc(-c2ccc(N3c4ccc(cc4)-c4ccccc4-c4ccc(cc4)N(c4ccc(-c5ccccn5)cc4)c4ccc(cc4)-c4ccccc4-c4ccc3cc4)cc2)nc1 ; c1ccc(-c2ccc(N3c4ccc(cc4)-c4ccccc4-c4ccc(cc4)N(c4ccc5c(c4)oc4ccccc45)c4ccc(cc4)-c4ccccc4-c4ccc3cc4)cc2)cc1]

In some embodiments, the material for an organic EL device according embodiments of to the present disclosure may include at least one selected from Compounds 24 to 28.

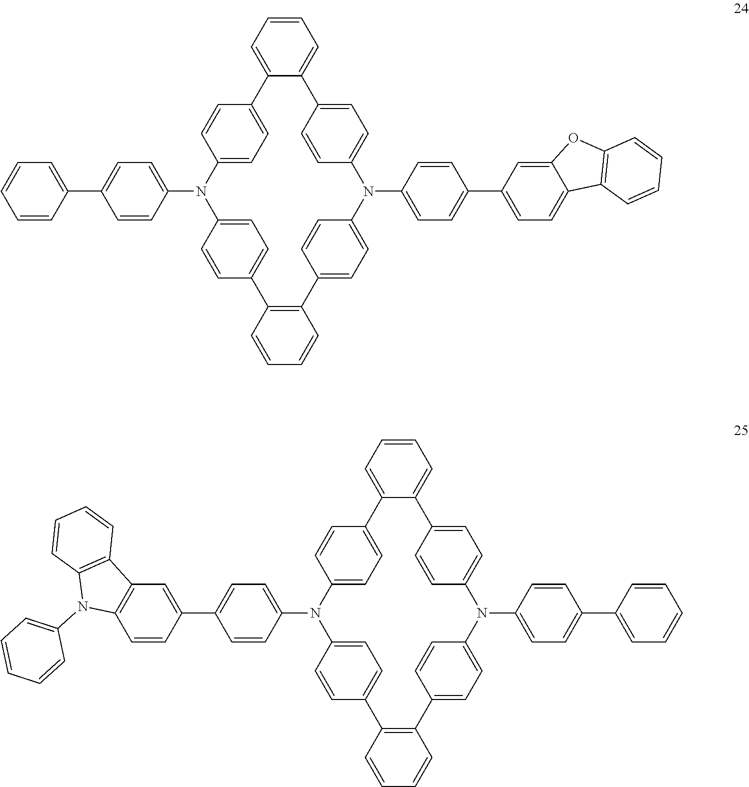 [SMILES: c1ccc(-c2ccc(N3c4ccc(cc4)-c4ccccc4-c4ccc(cc4)N(c4ccc(-c5ccc6c(c5)oc5ccccc56)cc4)c4ccc(cc4)-c4ccccc4-c4ccc3cc4)cc2)cc1 ; c1ccc(-c2ccc(N3c4ccc(cc4)-c4ccccc4-c4ccc(cc4)N(c4ccc(-c5ccc6c(c5)c5ccccc5n6-c5ccccc5)cc4)c4ccc(cc4)-c4ccccc4-c4ccc3cc4)cc2)cc1]

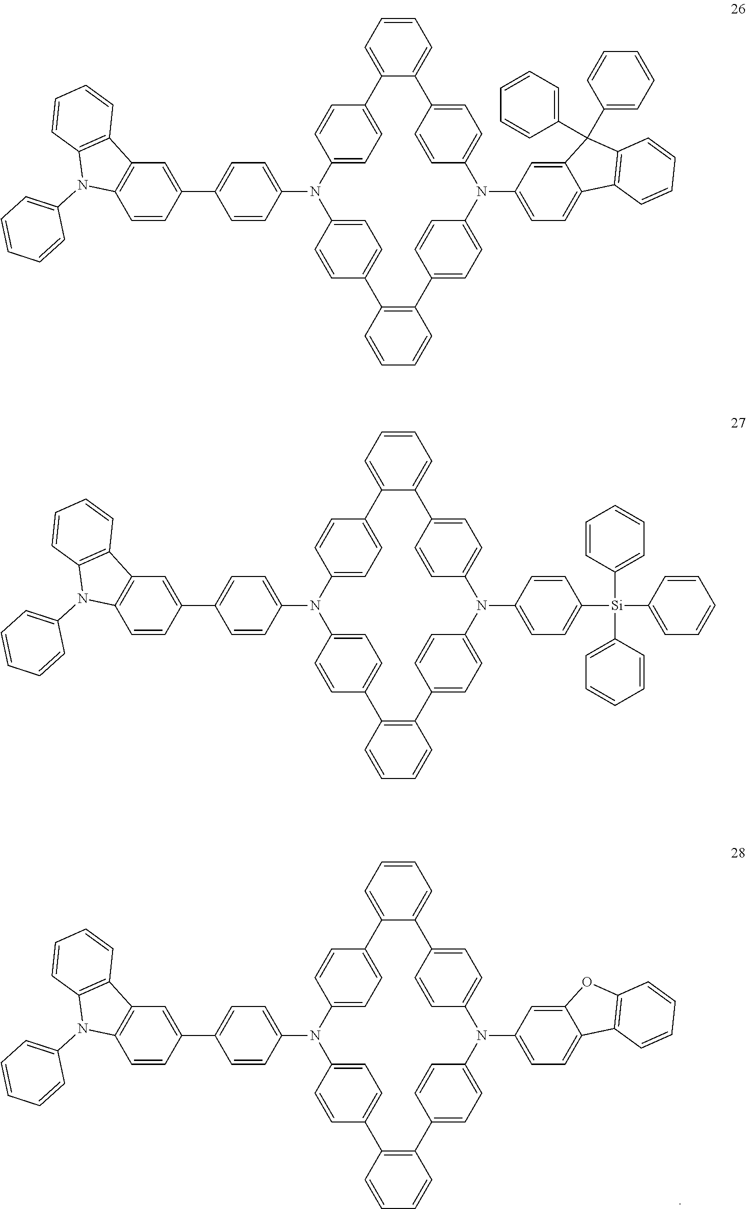 [SMILES: c1ccc(-n2c3ccccc3c3cc(-c4ccc(N5c6ccc(cc6)-c6ccccc6-c6ccc(cc6)N(c6ccc7c(c6)C(c6ccccc6)(c6ccccc6)c6ccccc6-7)c6ccc(cc6)-c6ccccc6-c6ccc5cc6)cc4)ccc32)cc1 ; c1ccc(-n2c3ccccc3c3cc(-c4ccc(N5c6ccc(cc6)-c6ccccc6-c6ccc(cc6)N(c6ccc([Si](c7ccccc7)(c7ccccc7)c7ccccc7)cc6)c6ccc(cc6)-c6ccccc6-c6ccc5cc6)cc4)ccc32)cc1 ; c1ccc(-n2c3ccccc3c3cc(-c4ccc(N5c6ccc(cc6)-c6ccccc6-c6ccc(cc6)N(c6ccc7c(c6)oc6ccccc67)c6ccc(cc6)-c6ccccc6-c6ccc5cc6)cc4)ccc32)cc1]

The material for an organic EL device according to embodiments of the present disclosure may be used in an emission layer of an organic device. In some embodiments, the material for an organic EL device according to embodiments of the present disclosure may be used in a layer selected from the stacked layers positioned between an emission layer and an anode. By using the material for an organic EL device according to embodiments of the present disclosure having a low HOMO level, carrier transport properties may be improved, hole mobility may be improved because of using diamine, and the organic EL device having high efficiency may be realized. In addition, since the material for an organic EL device according to embodiments of the present disclosure has a relatively wide (e.g., large) energy gap which may correspond to a blue region, application to green to red regions may also be possible.

### (Organic EL Device)

The organic EL device using the material for an organic EL device according to embodiments of the present disclosure will be explained with reference to the drawing. The drawing is a schematic view of the organic EL device 100 according to an embodiment of the present disclosure. The organic EL device 100 may include a substrate 102, an anode 104, a hole injection layer 106, a hole transport layer 108, an emission layer 110, an electron transport layer 112, an electron injection layer 114 and a cathode 116. In some embodiments, the material for an organic EL device according to embodiments of the present disclosure may be used in an emission layer 110. In addition, in some embodiments, the material for an organic EL device according to embodiments of the present disclosure may be used in a layer selected from the stacked layers positioned between the emission layer 110 and the anode 104.

For example, the material for an organic EL device according to embodiments of the present disclosure may be used in the hole transport layer 108. The substrate 102 may be a transparent glass substrate, a semiconductor substrate formed using silicon, and/or the like, a flexible substrate of a resin, and/or the like. The anode 104 may be positioned on the substrate 102 and may be formed using indium tin oxide (ITO), indium zinc oxide (IZO), and/or the like. The hole injection layer 106 may be positioned on the anode 104 and may include 4,4′,4″-tris[2-naphthyl(phenyl)amino]triphenylamine(2-TNATA), N,N,N′,N′-tetrakis(3-nnethylphenyl)-3,3′-dimethylbenzidine (HMTPD), and/or the like. The hole transport layer 108 may be positioned on the hole injection layer 106 and may be formed using the material for an organic EL device according to embodiments of the present disclosure. The emission layer 110 may be positioned on the hole transport layer 108 and may be formed using the material for an organic EL device according to embodiments of the present disclosure. In addition, the emission layer may include a host material including, for example, 9,10-di(2-naphthyl)anthracene (ADN) doped with 2,5,8,11-tetra-t-butylperylene(TBP). The electron transport layer 112 may be positioned on the emission layer 110 and may be formed using a material including, for example, tris(8-hydroxyquinolinato)aluminum (Alq3). The electron injection layer 114 may be positioned on the electron transport layer 112 and may be formed using, for example, a material including lithium fluoride (LiF). The cathode 116 may be positioned on the electron injection layer 114 and may be formed using a metal such as Al or a transparent material such as ITO and/or IZO. Each of the above-described thin layers may be formed by one or more suitable forming methods (according to the material used to form the layer) such as a vacuum evaporation method, a sputtering method, various coating methods, and/or the like.

In the organic EL device 100 according to an embodiment of the present disclosure, a hole transport layer capable of realizing high emission efficiency may be formed by using the above-described material for an organic EL device according to embodiments of the present disclosure. For example, the material for an organic EL device according to embodiments of the present disclosure may be utilized in an organic EL display including an active matrix using a thin film transistor (TFT).

In the organic EL device 100 according to embodiments of the present disclosure, high emission efficiency of the organic EL device may be realized by using the material for an organic EL device according to embodiments of the present disclosure in an emission layer or a layer selected from the stacked layers positioned between the emission layer and the anode of the organic EL device.

### EXAMPLES

### (Preparation Method)

The above-described material for an organic EL device according to embodiments of the present disclosure may be synthesized, for example, according the following method.

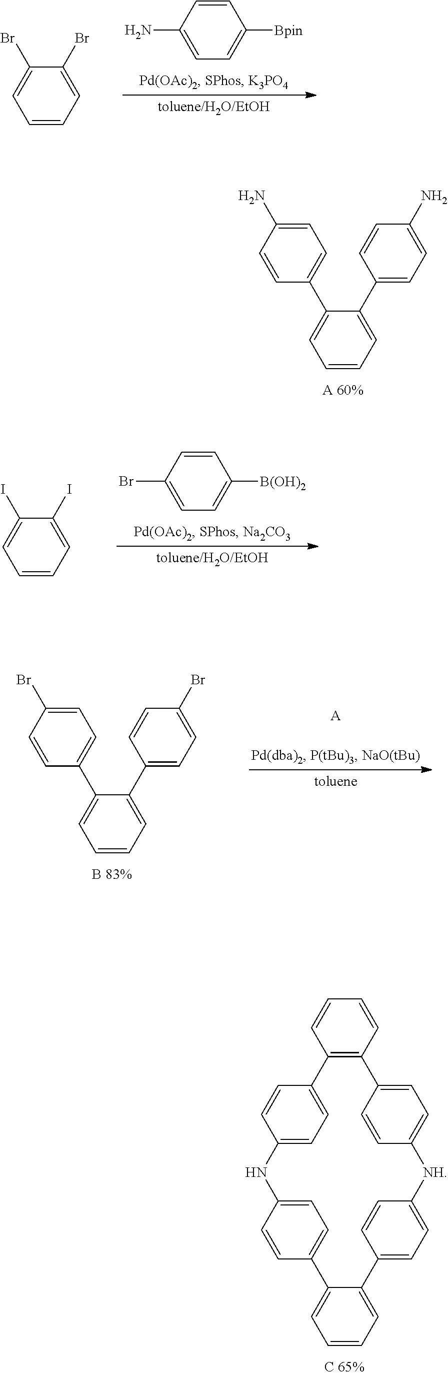

### (Synthesis of Compound A)

Under an argon atmosphere, 10.0 g of 1,2-dibromobenzene, 18.6 g of 4-(4,4,5,5-tetramethyl-1,3,2-dioxaborolane-2-yl)aniline

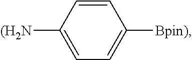

0.95 g of palladium acetate, 3.48 g of 2-dicyclohexylphosphino-2′,6′-bimethoxybiphenyl (SPhos), 36.0 g of tripotassium phosphate were added to a 1 L, three necked flask and stirred in 400 mL of a mixture solvent of toluene, water and ethanol (volume ratio of 10:1:1) at about 100° C. for about 24 hours. Water was added to the resulting reaction solution; an organic layer was separated therefrom, and solvent was distilled. The crude product thus obtained was separated by silica gel column chromatography (using dichloromethane as a solvent) to produce 6.62 g of Compound A as a white solid (Yield 60%). The molecular weight of the obtained material measured by Fast Atom Bombardment-Mass Spectrometry (FAB-MS) was about 260, and the chemical formula thereof was proposed to be C₁₈H₁₆N₂, and the target material was confirmed to be Compound A.

### (Synthesis of Compound B)

Under an argon atmosphere, 10.0 g of 1,2-diiodobenzne, 12.2 g of 4-bromophenyl boronic acid, 0.68 g of palladium acetate, 2.49 g of 2-dicyclohexylphosphino-2′,6′-bimethoxybiphenyl (SPhos) and 12.9 g of sodium carbonate were added to a 1 L, three necked flask and was stirred in 400 mL of a mixture solvent of toluene, water and ethanol (volume ratio of 10:1:1) at about 100° C. for about 24 hours. Water was added to the resulting reaction solution to separate an organic layer, and solvent was distilled. The crude product thus obtained was separated by silica gel column chromatography (using toluene and hexane as solvents) to produce 9.76 g of Compound B as a white solid (Yield 83%). The molecular weight of the obtained material measured by FAB-MS was about 387, and the chemical formula thereof was proposed to be C₁₈H₁₂Br₂, and the target material was confirmed to be Compound B.

### (Synthesis of Compound C)

Under an argon atmosphere, 5.00 g of Compound A, 7.45 g of Compound B, 0.55 g of bis(dibenzylidene acetone)palladium(0) (Pd(dba)₂), 0.77 g of tri-tert-butylphosphine and 2.77 g of sodium tert-butoxide were added to a 500 mL, three necked flask, followed by heating and refluxing in 200 mL of a toluene solvent for about 8 hours. After air cooling, water was added to the resulting reaction solution to separate an organic layer, and solvent was distilled. The crude product thus obtained was separated by silica gel column chromatography (using toluene as a solvent) to produce 6.07 g of Compound C as a white solid (Yield 65%). The molecular weight of the obtained material measured by FAB-MS was about 486, and the chemical formula thereof was proposed to be C₃₆H₂₆N₂, and the target material was confirmed to be Compound C.

### (Synthesis of Compound D)

By using the Compound C obtained according to the synthesis method described above as a starting material, Compound D was synthesized as follows:

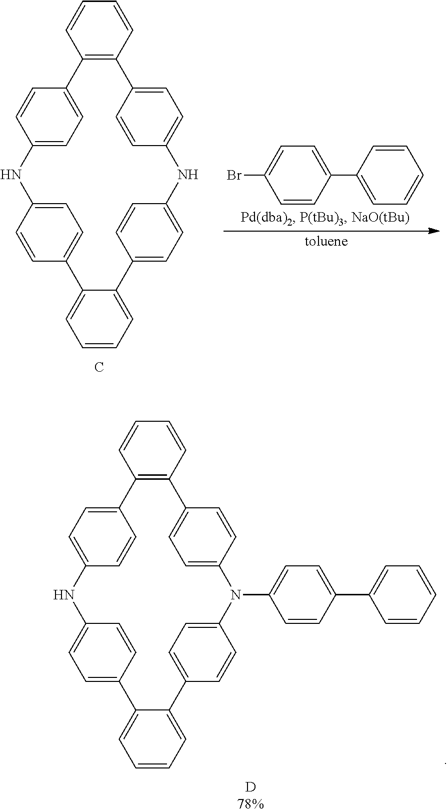

Under an argon atmosphere, 2.00 g of Compound C, 0.96 g of 4-bromobiphenyl, 0.12 g of bis(dibenzylideneacetone)palladium(0), 0.17 g of tri-tert-butylphosphine and 0.59 g of sodium tert-butoxide were added to a 200 mL, three necked flask, followed by heating and refluxing in 40 mL of a toluene solvent for about 4 hours. After air cooling, water was added to the resulting reaction solution to separate an organic layer, and solvent was distilled. The crude product thus obtained was separated by silica gel column chromatography (using toluene and hexane as solvents) to produce 2.05 g of Compound D as a white solid (Yield 78%). The molecular weight of the obtained material measured by FAB-MS was about 638, and the chemical formula thereof was proposed to be C₄₈H₃₄N₂, and the target material was confirmed to be Compound D.

(Synthesis of Compound 2)

By using Compound C as a starting material, Compound 2 was synthesized as follows:

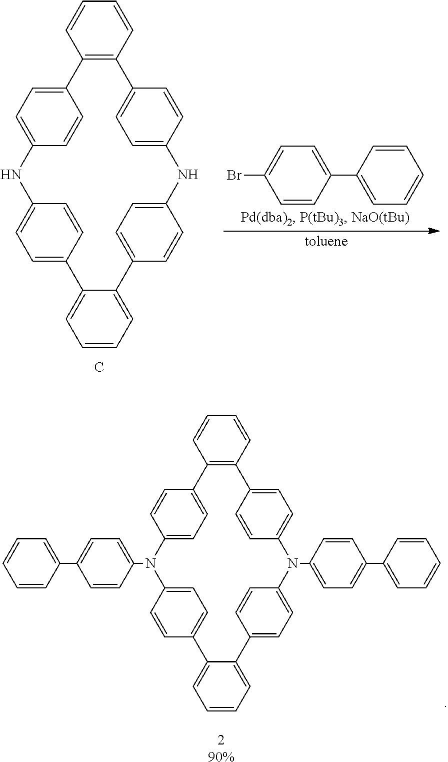

Under an argon atmosphere, 1.00 g of Compound C, 0.96 g of 4-bromobiphenyl, 0.06 g of bis(dibenzylideneacetone)palladium(0), 0.16 g of tri-tert-butylphosphine and 0.30 g of sodium tert-butoxide were added to a 100 mL of three necked flask, followed by heating and refluxing in 30 mL of a toluene solvent for about 6 hours. After air cooling, water was added to the resulting reaction solution to separate an organic layer, and solvent was distilled. The crude product thus obtained was separated by silica gel column chromatography (using toluene and hexane as solvents) to produce 1.46 g of Compound 2 as a white solid (Yield 90%).

The chemical shift Values of the obtained material measured by ¹H NMR (300 MHz, DMSO-d₆) were 7.75-7.34(m, 18H), 7.23(d, J=8.3 Hz, 4H), 7.02(d, J=8.2 Hz, 8H), 6.75(d, J=8.3 Hz, 4H), 6.64(d, J=8.2 Hz, 8H). The molecular weight of the obtained material measured by FAB-MS was about 790, and the chemical formula thereof was proposed to be C₆₀H₄₂N₂, and the target material was confirmed to be Compound 2.

### (Synthesis of Compound 10)

Compound 10 was synthesized in substantially the same manner as Compound 2 and using Compound C as a starting material, except that 3-bromodibenzofuran was used instead of 4-bromobiphenyl. The molecular weight of the obtained material measured by FAB-MS was about 818, and the chemical formula thereof was proposed to be C₆₀H₃₈N₂O₂, and the target material was confirmed to be Compound 10.

### (Synthesis of Compound 24)

By using Compound D as a starting material, Compound 24 was synthesized as follows:

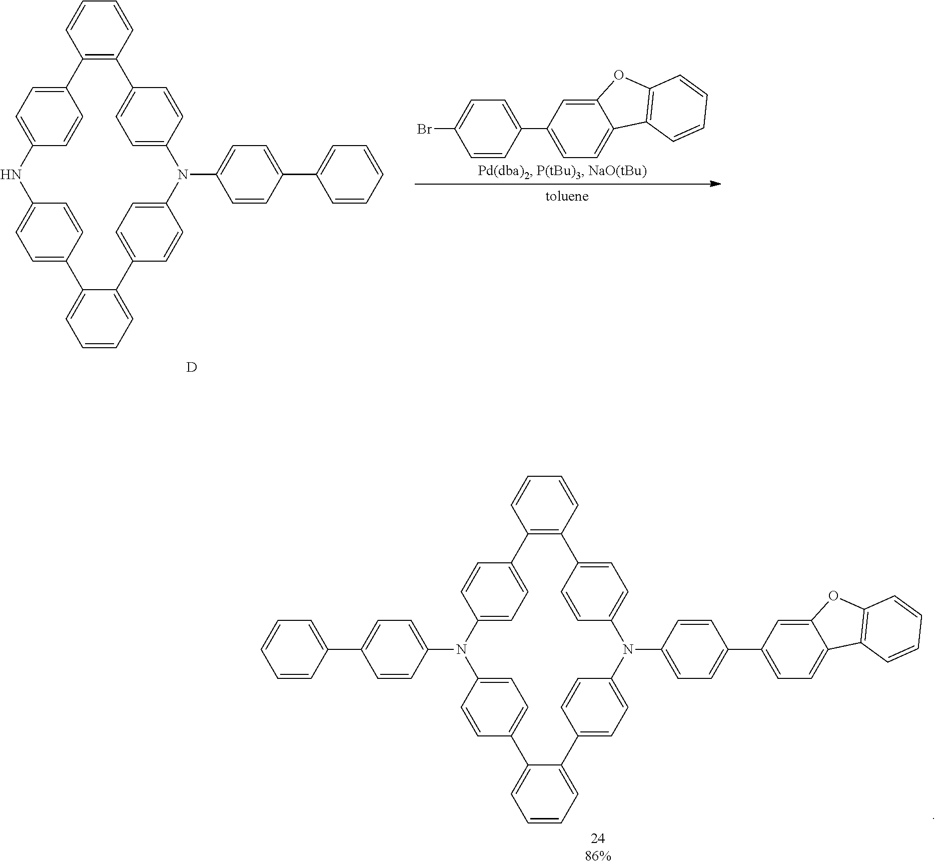

Under an argon atmosphere, 1.00 g of Compound D, 0.51 g of 3-(4-bromophenyl)dibenzofuran, 0.05 g of bis(dibenzylideneacetone)palladium(0), 0.06 g of tri-tert-butylphosphine and 0.23 g of sodium tert-butoxide were added to a 100 mL, three necked flask, followed by heating and refluxing in 30 mL of a toluene solvent for about 6 hours. After air cooling, water was added to the resulting reaction mixture to separate an organic layer, and solvent was distilled. The crude product thus obtained was separated by silica gel column chromatography (using toluene and hexane as solvents) to produce 1.19 g of Compound 24 as a white solid (Yield 86%). The molecular weight of the obtained material measured by FAB-MS was about 880, and the chemical formula thereof was proposed to be C₆₆H₄₄N₂O, and the target material was confirmed to be Compound 24.

### (Synthesis of Compound 25)

Compound 25 was synthesized in substantially the same manner as Compound 24 and using Compound D as a starting material, except that 2-(4-bromophenyl)-9-phenylcarbazole was used instead of 3-(4-bromophenyl)dibenzofuran. The molecular weight of the obtained material measured by FAB-MS was about 955, and the chemical formula thereof was proposed to be C₇₂H₄₉N₃, and the target material was confirmed to be Compound 25.

### (Measuring of HOMO Level)

For Compounds 2, 10, 24 and 25, and Comparative Compounds C1 and C2 (illustrated below), HOMO levels were measured.

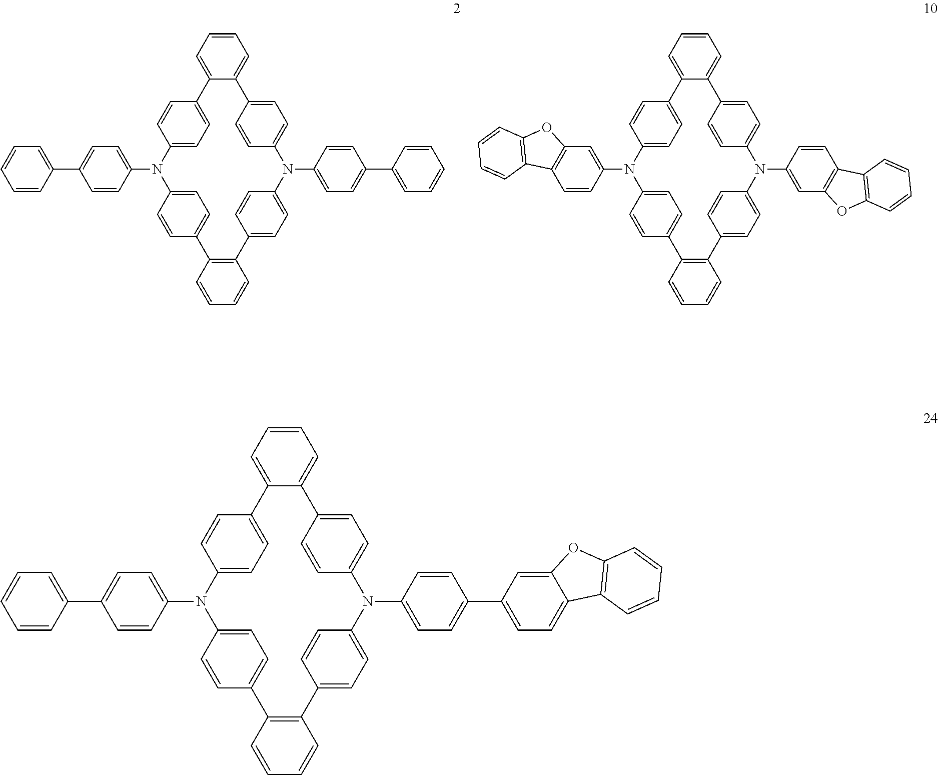 [SMILES: c1ccc(-c2ccc(N3c4ccc(cc4)-c4ccccc4-c4ccc(cc4)N(c4ccc(-c5ccccc5)cc4)c4ccc(cc4)-c4ccccc4-c4ccc3cc4)cc2)cc1 ; c1ccc2c(c1)-c1ccc(cc1)N(c1ccc3c(c1)oc1ccccc13)c1ccc(cc1)-c1ccccc1-c1ccc(cc1)N(c1ccc3c(c1)oc1ccccc13)c1ccc-2cc1 ; c1ccc(-c2ccc(N3c4ccc(cc4)-c4ccccc4-c4ccc(cc4)N(c4ccc(-c5ccc6c(c5)oc5ccccc56)cc4)c4ccc(cc4)-c4ccccc4-c4ccc3cc4)cc2)cc1]

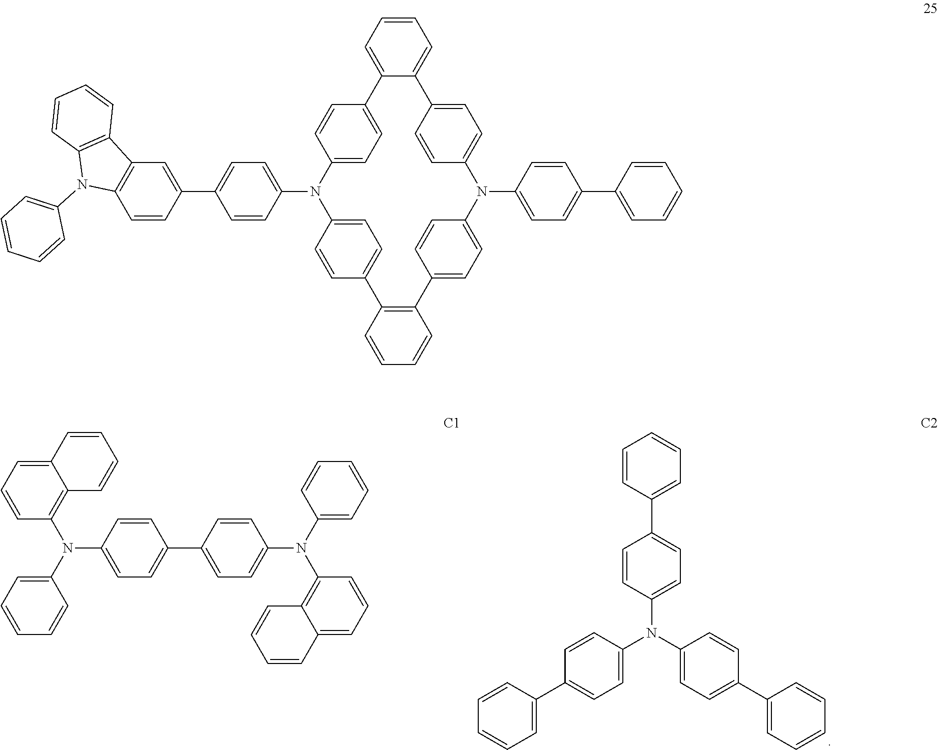 [SMILES: c1ccc(-c2ccc(N3c4ccc(cc4)-c4ccccc4-c4ccc(cc4)N(c4ccc(-c5ccc6c(c5)c5ccccc5n6-c5ccccc5)cc4)c4ccc(cc4)-c4ccccc4-c4ccc3cc4)cc2)cc1 ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccccc4)cc3)cc2)cc1]

The measuring of the HOMO level was conducted using a photoelectronic spectrophotometer AC-3 (manufactured by RIKEN KEIKI Co., Ltd.) in the air. The results of HOMO level measurements are shown in Table 1.

TABLE 1

| Compound | HOMO (eV) |
|---|---|
| Compound 2 | −5.82 |
| Compound 10 | −5.76 |
| Compound 24 | −5.80 |
| Compound 25 | −5.74 |
| Comparative Compound C1 | −5.54 |
| Comparative Compound C2 | −5.75 |

### Examples 1 to 4 and Comparative Examples 1 and 2

Organic EL devices according to Examples 1 to 4 were manufactured by respectively using Compounds 2, 10, 24 and 25 synthesized according to the above-described method as a hole transport material. In addition, organic EL devices according to Comparative Examples 1 and 2 were manufactured by respectively using Comparative Compounds C1 and C2.

For each of the organic EL devices of Examples 1 to 4 and Comparative Examples 1 and 2, a transparent glass substrate was used as the substrate, the anode was formed using ITO with a thickness of about 150 nm, the hole injection layer was formed using TNATA to a layer thickness of about 60 nm, the hole transport layer (HTL) was formed using the corresponding compound selected from Compounds 2, 10, 24, 25, and Comparative Compounds C1 and C2 to a layer thickness of about 30 nm, the emission layer was formed using ADN doped with 3% TBP to a layer thickness of about 25 nm, the electron transport layer was formed using Alq3 to a layer thickness of about 25 nm, the electron injection layer was formed using LiF to a layer thickness of about 1 nm, and the cathode was formed using Al to a layer thickness of about 100 nm.

For each of the organic EL devices thus manufactured, voltage and emission efficiency were evaluated. For the evaluation of the emission properties of the organic EL devices, a brightness light distribution characteristics measurement system C9920-11 manufactured by Hamamatsu Photonics Co. was used, and current density was set to 10 mA/cm².

TABLE 2

| | | Current | | Emission |
|---|---|---|---|---|
| | | density | | efficiency |
| Example | HTL | (mA/cm²) | Voltage (V) | (cd/A) |
| Example 1 | Compound 2 | 10 | 7.4 | 7.0 |
| Example 2 | Compound 10 | 10 | 7.3 | 7.3 |
| Example 3 | Compound 24 | 10 | 7.1 | 7.5 |
| Example 4 | Compound 25 | 10 | 7.2 | 7.1 |
| Comparative | Comparative | 10 | 8.1 | 5.3 |
| Example 1 | Compound C1 | | | |
| Comparative | Comparative | 10 | 7.5 | 5.2 |
| Example 2 | Compound C2 | | | |

From the results shown in Table 2, it can be seen that the organic EL devices of Examples 1 to 4 exhibited decreased voltage and increased emission efficiency, when compared with the organic EL devices of Comparative Examples 1 and 2. Without being bound by any particular theory, it is believed that since the HOMO levels of compounds used in Examples 1 to 4 was lower than that of the compound used in Comparative Example 1 (as shown in Table 1), driving voltage of the organic EL devices of Examples 1 to 4 was decreased, and thus carrier transport properties of the organic EL devices were improved. Without being bound by any particular theory, it is further believed that the improved emission efficiency of the organic EL devices according to Examples 1 to 4, as compared to that of the organic EL device according to Comparative Example 2, was achieved by including a diamine compound, and thus increasing hole mobility.

As described above, high emission efficiency of an organic EL device may be achieved in a blue region by using the material for an organic EL device according to embodiments of the present disclosure. Since the material for an organic EL device has a relatively wide (e.g., large) energy gap which may correspond to a blue region, application to green to red regions may be also possible.

As used herein, expressions such as “at least one selected from,” “one selected from,” “at least one of,” and “one of,” when preceding a list of elements, modify the entire list of elements and do not modify the individual elements of the list. Further, the use of “may” when describing embodiments of the present invention refers to “one or more embodiments of the present invention.”

In addition, as used herein, the terms “use,” “using,” and “used” may be considered synonymous with the terms “utilize,” “utilizing,” and “utilized,” respectively. Also, the terms “substantially,” “about,” and similar terms are used as terms of approximation and not as terms of degree, and are intended to account for the inherent deviations in measured or calculated values that would be recognized by those of ordinary skill in the art.

It will be understood that the terms, such as “comprises,” “comprising,” “includes”, and/or “including”, when used in this specification, specify the presence of stated features, integers, steps, operations, elements, and/or components, but do not preclude the presence or addition of one or more other features, integers, steps, operations, elements, components, and/or groups thereof.

It will be further understood that any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112(a) and 35 U.S.C. §132(a).

The above-disclosed subject matter is to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent (EL) device represented by Formula (1): 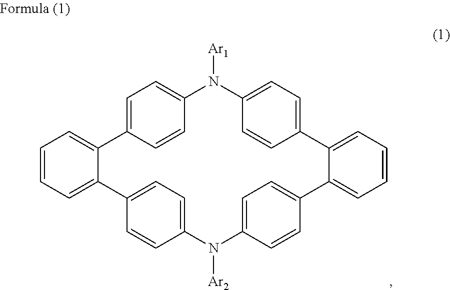
wherein, in Formula (1), Ar₁ and Arₑ are each independently selected from a substituted or unsubstituted aryl group and a substituted or unsubstituted heteroaryl group.

2. The material of claim 1, wherein the material comprises at least one selected from Compounds 1 to 28:

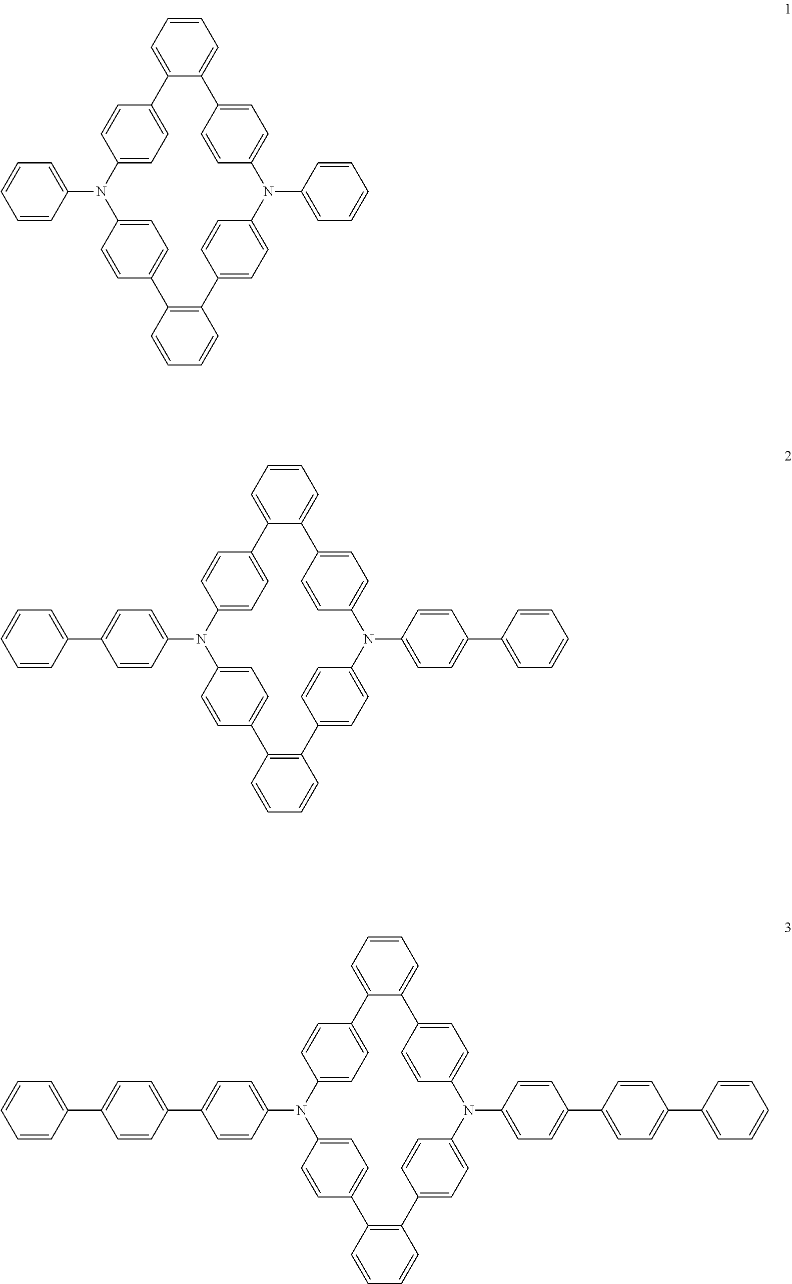 [SMILES: c1ccc(N2c3ccc(cc3)-c3ccccc3-c3ccc(cc3)N(c3ccccc3)c3ccc(cc3)-c3ccccc3-c3ccc2cc3)cc1 ; c1ccc(-c2ccc(N3c4ccc(cc4)-c4ccccc4-c4ccc(cc4)N(c4ccc(-c5ccccc5)cc4)c4ccc(cc4)-c4ccccc4-c4ccc3cc4)cc2)cc1 ; c1ccc(-c2ccc(-c3ccc(N4c5ccc(cc5)-c5ccccc5-c5ccc(cc5)N(c5ccc(-c6ccc(-c7ccccc7)cc6)cc5)c5ccc(cc5)-c5ccccc5-c5ccc4cc5)cc3)cc2)cc1]

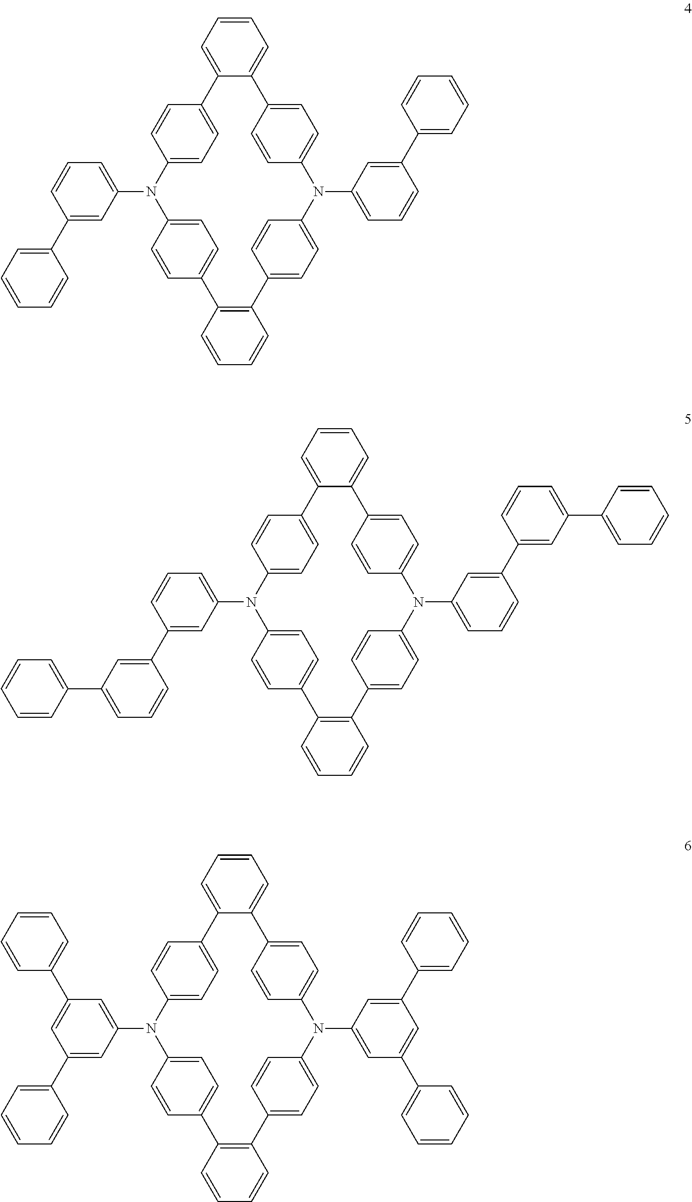 [SMILES: c1ccc(-c2cccc(N3c4ccc(cc4)-c4ccccc4-c4ccc(cc4)N(c4cccc(-c5ccccc5)c4)c4ccc(cc4)-c4ccccc4-c4ccc3cc4)c2)cc1 ; c1ccc(-c2cccc(-c3cccc(N4c5ccc(cc5)-c5ccccc5-c5ccc(cc5)N(c5cccc(-c6cccc(-c7ccccc7)c6)c5)c5ccc(cc5)-c5ccccc5-c5ccc4cc5)c3)c2)cc1 ; c1ccc(-c2cc(-c3ccccc3)cc(N3c4ccc(cc4)-c4ccccc4-c4ccc(cc4)N(c4cc(-c5ccccc5)cc(-c5ccccc5)c4)c4ccc(cc4)-c4ccccc4-c4ccc3cc4)c2)cc1]

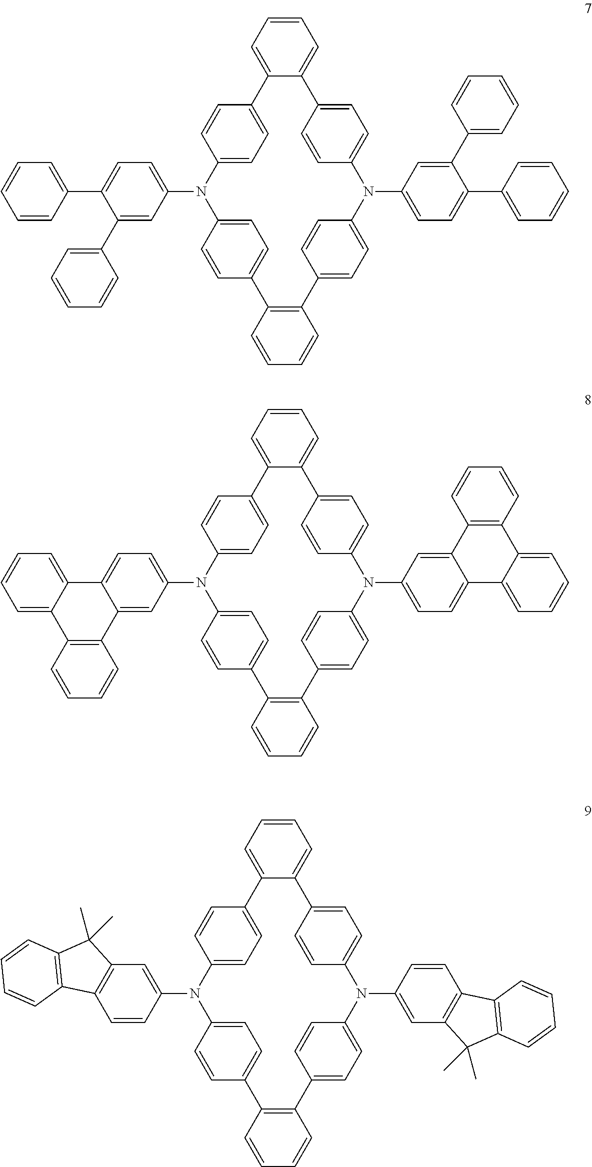

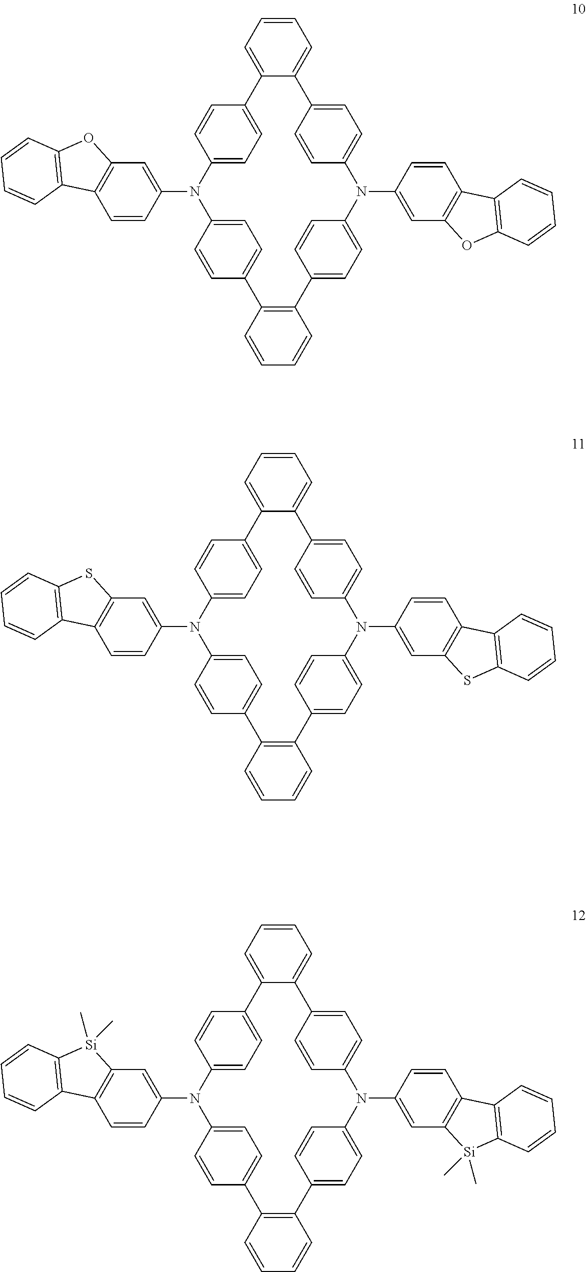 [SMILES: c1ccc2c(c1)-c1ccc(cc1)N(c1ccc3c(c1)oc1ccccc13)c1ccc(cc1)-c1ccccc1-c1ccc(cc1)N(c1ccc3c(c1)oc1ccccc13)c1ccc-2cc1 ; c1ccc2c(c1)-c1ccc(cc1)N(c1ccc3c(c1)sc1ccccc13)c1ccc(cc1)-c1ccccc1-c1ccc(cc1)N(c1ccc3c(c1)sc1ccccc13)c1ccc-2cc1 ; C[Si]1(C)c2ccccc2-c2ccc(N3c4ccc(cc4)-c4ccccc4-c4ccc(cc4)N(c4ccc5c(c4)[Si](C)(C)c4ccccc4-5)c4ccc(cc4)-c4ccccc4-c4ccc3cc4)cc21]

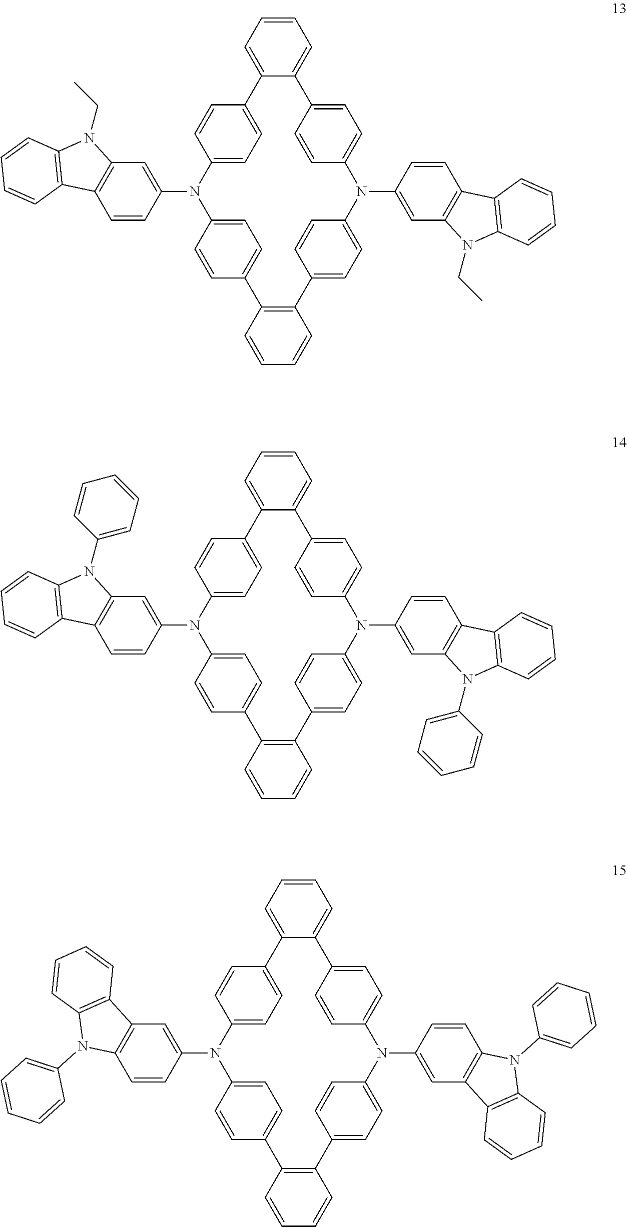 [SMILES: CCn1c2ccccc2c2ccc(N3c4ccc(cc4)-c4ccccc4-c4ccc(cc4)N(c4ccc5c6ccccc6n(CC)c5c4)c4ccc(cc4)-c4ccccc4-c4ccc3cc4)cc21 ; c1ccc(-n2c3ccccc3c3ccc(N4c5ccc(cc5)-c5ccccc5-c5ccc(cc5)N(c5ccc6c7ccccc7n(-c7ccccc7)c6c5)c5ccc(cc5)-c5ccccc5-c5ccc4cc5)cc32)cc1 ; c1ccc(-n2c3ccccc3c3cc(N4c5ccc(cc5)-c5ccccc5-c5ccc(cc5)N(c5ccc6c(c5)c5ccccc5n6-c5ccccc5)c5ccc(cc5)-c5ccccc5-c5ccc4cc5)ccc32)cc1]

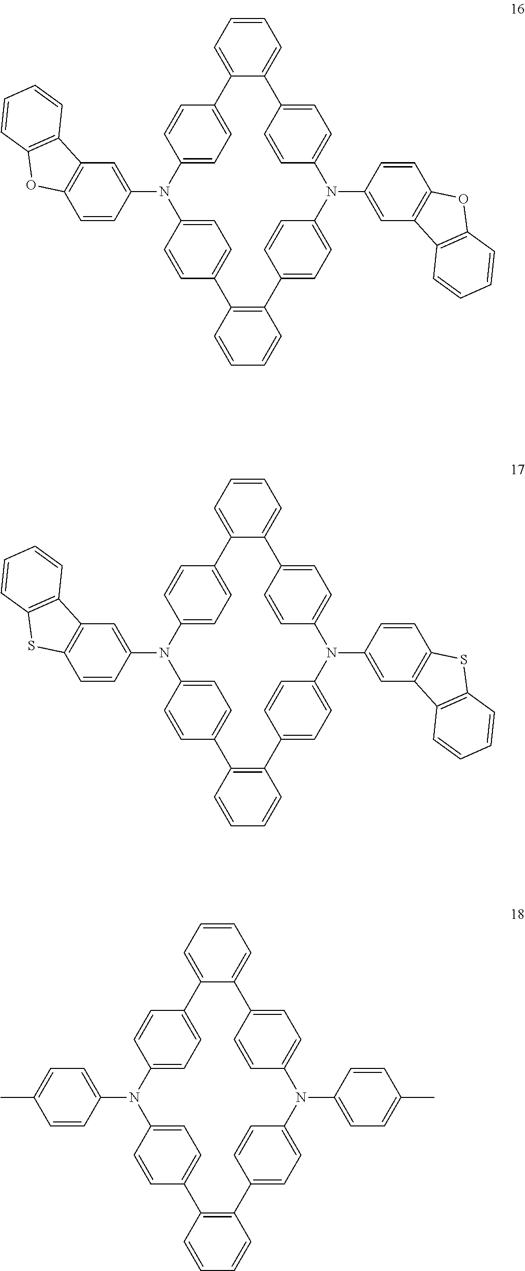

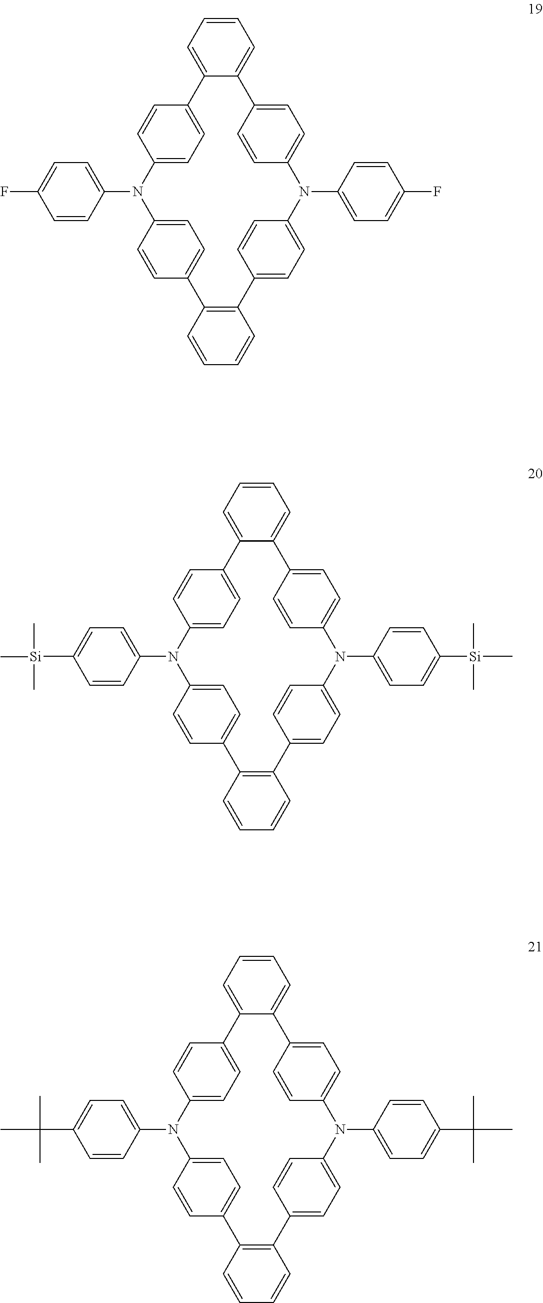

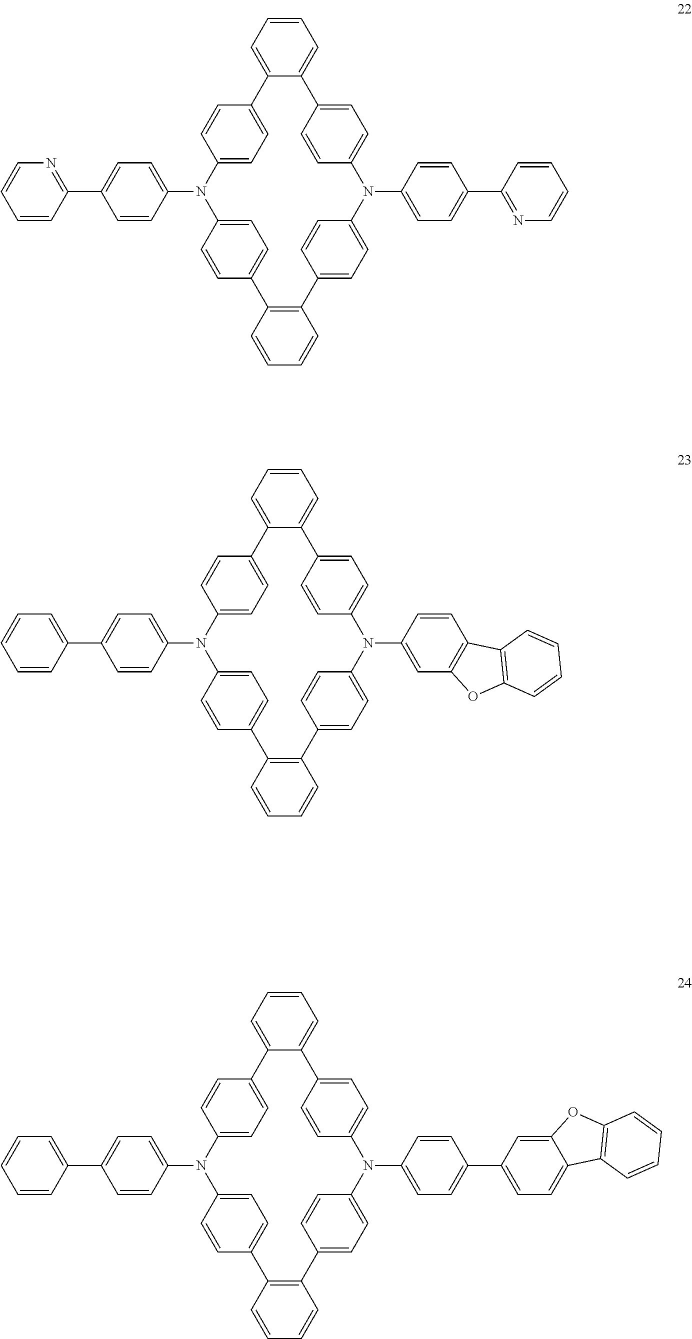 [SMILES: c1ccc(-c2ccc(N3c4ccc(cc4)-c4ccccc4-c4ccc(cc4)N(c4ccc(-c5ccccn5)cc4)c4ccc(cc4)-c4ccccc4-c4ccc3cc4)cc2)nc1 ; c1ccc(-c2ccc(N3c4ccc(cc4)-c4ccccc4-c4ccc(cc4)N(c4ccc5c(c4)oc4ccccc45)c4ccc(cc4)-c4ccccc4-c4ccc3cc4)cc2)cc1 ; c1ccc(-c2ccc(N3c4ccc(cc4)-c4ccccc4-c4ccc(cc4)N(c4ccc(-c5ccc6c(c5)oc5ccccc56)cc4)c4ccc(cc4)-c4ccccc4-c4ccc3cc4)cc2)cc1]

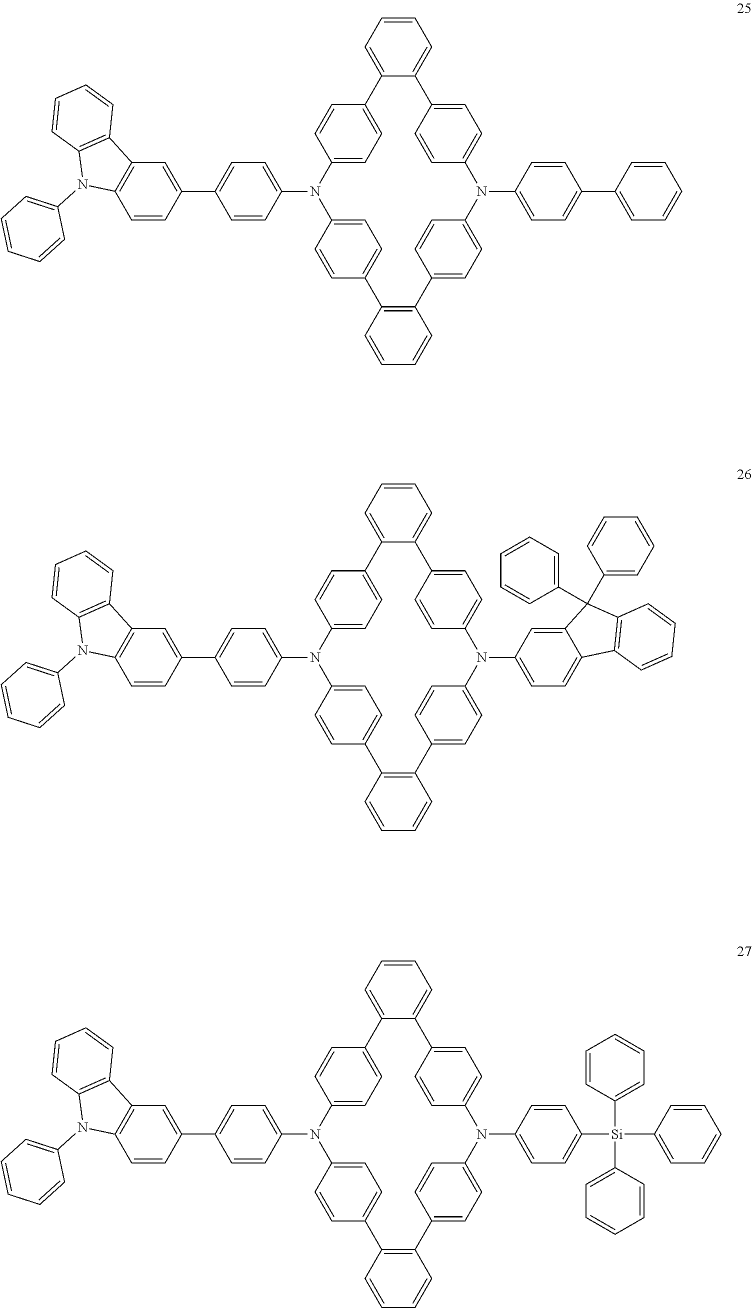 [SMILES: c1ccc(-c2ccc(N3c4ccc(cc4)-c4ccccc4-c4ccc(cc4)N(c4ccc(-c5ccc6c(c5)c5ccccc5n6-c5ccccc5)cc4)c4ccc(cc4)-c4ccccc4-c4ccc3cc4)cc2)cc1 ; c1ccc(-n2c3ccccc3c3cc(-c4ccc(N5c6ccc(cc6)-c6ccccc6-c6ccc(cc6)N(c6ccc7c(c6)C(c6ccccc6)(c6ccccc6)c6ccccc6-7)c6ccc(cc6)-c6ccccc6-c6ccc5cc6)cc4)ccc32)cc1 ; c1ccc(-n2c3ccccc3c3cc(-c4ccc(N5c6ccc(cc6)-c6ccccc6-c6ccc(cc6)N(c6ccc([Si](c7ccccc7)(c7ccccc7)c7ccccc7)cc6)c6ccc(cc6)-c6ccccc6-c6ccc5cc6)cc4)ccc32)cc1]

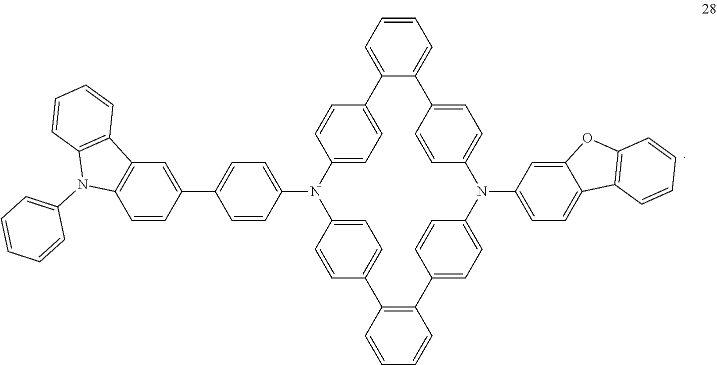 [SMILES: c1ccc(-n2c3ccccc3c3cc(-c4ccc(N5c6ccc(cc6)-c6ccccc6-c6ccc(cc6)N(c6ccc7c(c6)oc6ccccc67)c6ccc(cc6)-c6ccccc6-c6ccc5cc6)cc4)ccc32)cc1]

3. An organic electroluminescent (EL) device comprising:
an anode,
a cathode,
an emission layer between the anode and the cathode, and
a plurality of stacked layers between the emission layer and the anode,
wherein at least one layer of the plurality of stacked layers comprises a material for an organic EL device represented by Formula (1): 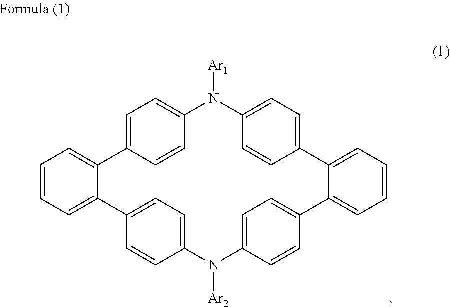
wherein, in Formula (1), Ar₁ and Ar₂ are each independently selected from a substituted or unsubstituted aryl group and a substituted or unsubstituted heteroaryl group.

4. The organic EL device of claim 3, wherein the material represented by Formula (1) comprises at least one selected from Compounds 1 to 28:

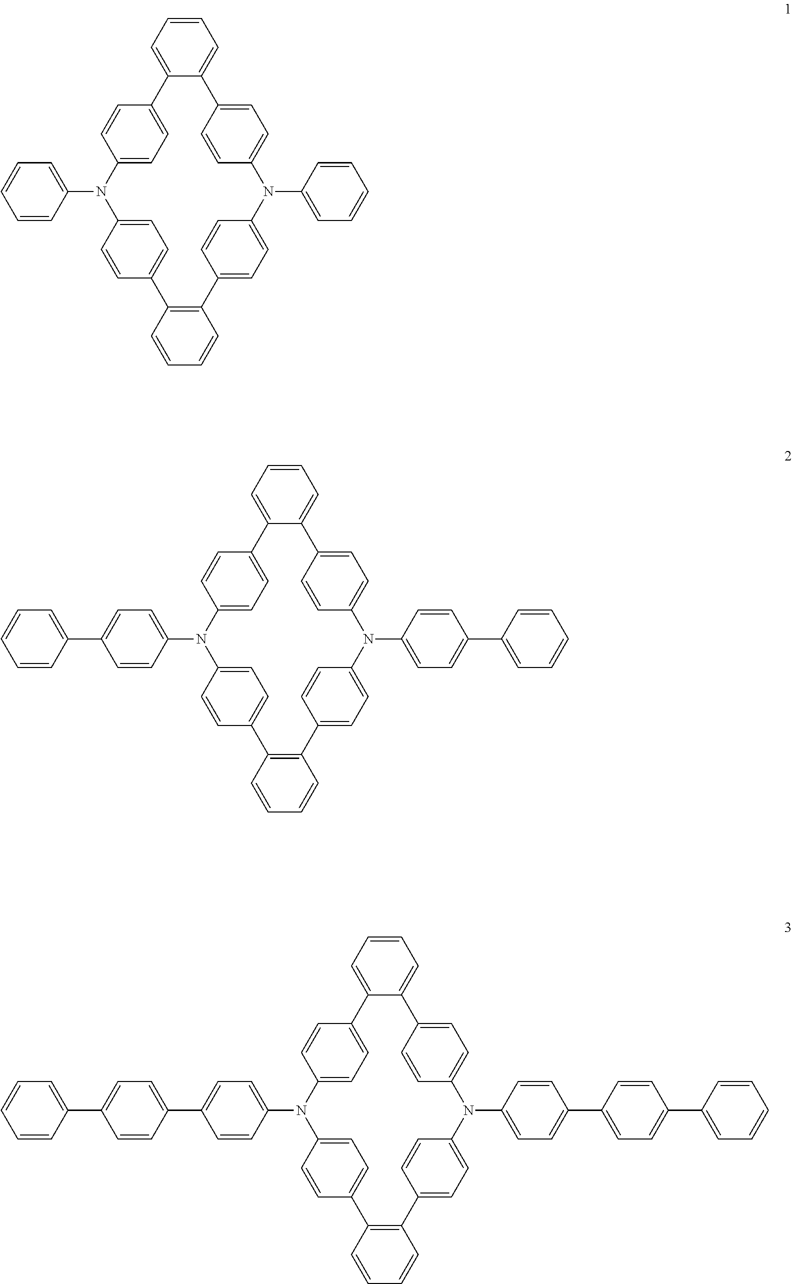 [SMILES: c1ccc(N2c3ccc(cc3)-c3ccccc3-c3ccc(cc3)N(c3ccccc3)c3ccc(cc3)-c3ccccc3-c3ccc2cc3)cc1 ; c1ccc(-c2ccc(N3c4ccc(cc4)-c4ccccc4-c4ccc(cc4)N(c4ccc(-c5ccccc5)cc4)c4ccc(cc4)-c4ccccc4-c4ccc3cc4)cc2)cc1 ; c1ccc(-c2ccc(-c3ccc(N4c5ccc(cc5)-c5ccccc5-c5ccc(cc5)N(c5ccc(-c6ccc(-c7ccccc7)cc6)cc5)c5ccc(cc5)-c5ccccc5-c5ccc4cc5)cc3)cc2)cc1]

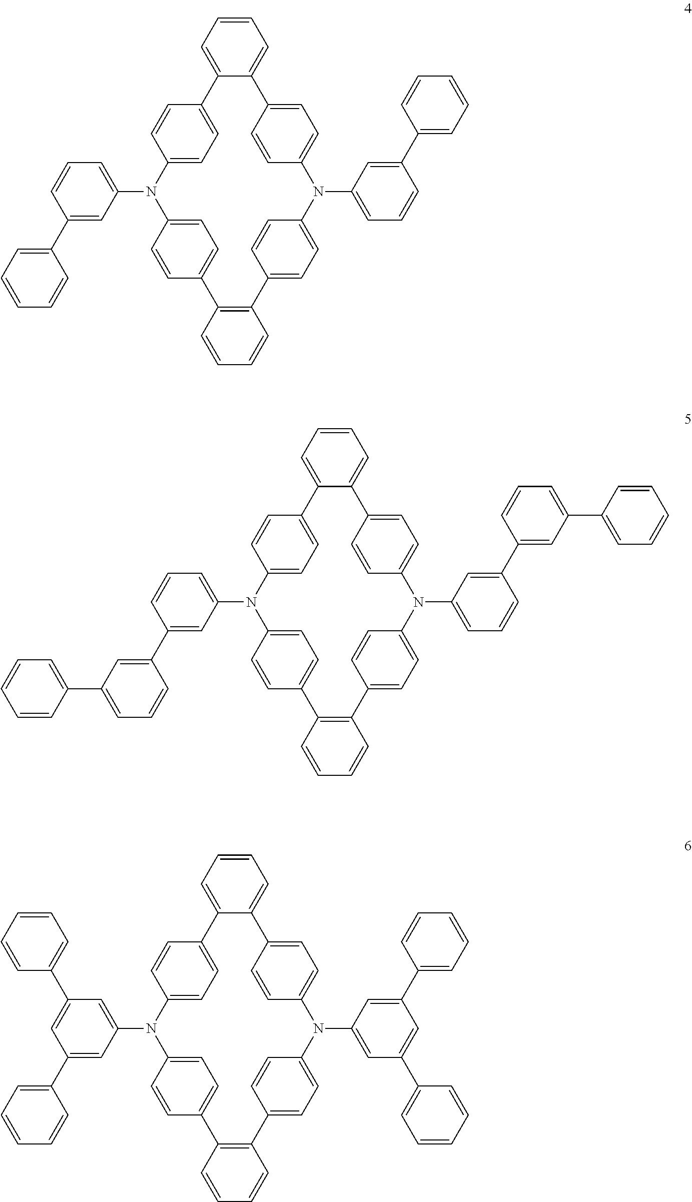 [SMILES: c1ccc(-c2cccc(N3c4ccc(cc4)-c4ccccc4-c4ccc(cc4)N(c4cccc(-c5ccccc5)c4)c4ccc(cc4)-c4ccccc4-c4ccc3cc4)c2)cc1 ; c1ccc(-c2cccc(-c3cccc(N4c5ccc(cc5)-c5ccccc5-c5ccc(cc5)N(c5cccc(-c6cccc(-c7ccccc7)c6)c5)c5ccc(cc5)-c5ccccc5-c5ccc4cc5)c3)c2)cc1 ; c1ccc(-c2cc(-c3ccccc3)cc(N3c4ccc(cc4)-c4ccccc4-c4ccc(cc4)N(c4cc(-c5ccccc5)cc(-c5ccccc5)c4)c4ccc(cc4)-c4ccccc4-c4ccc3cc4)c2)cc1]

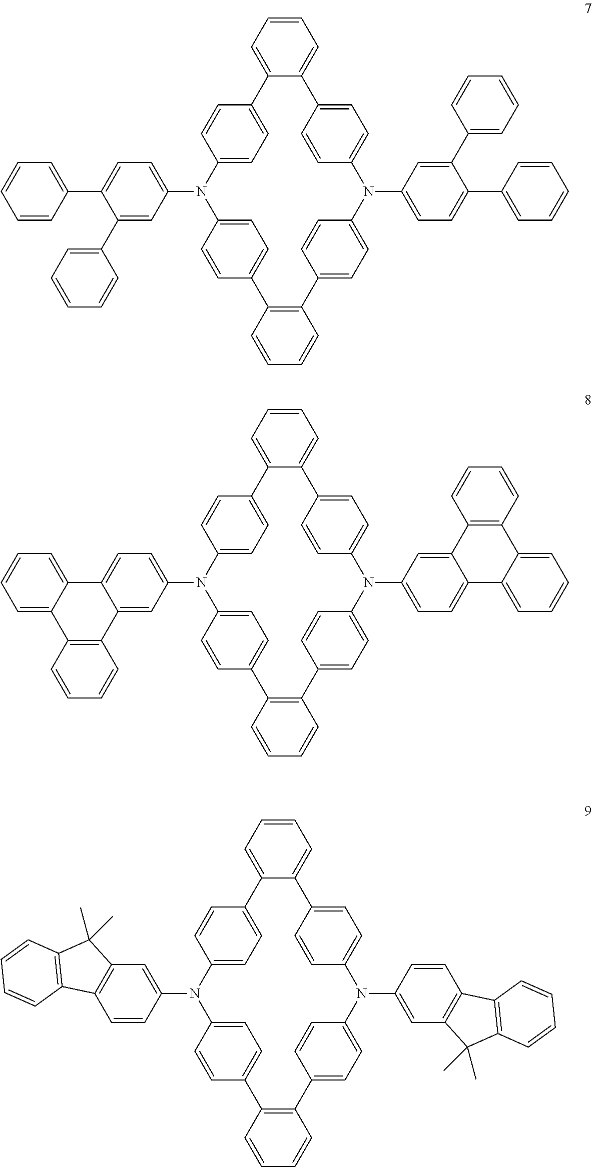

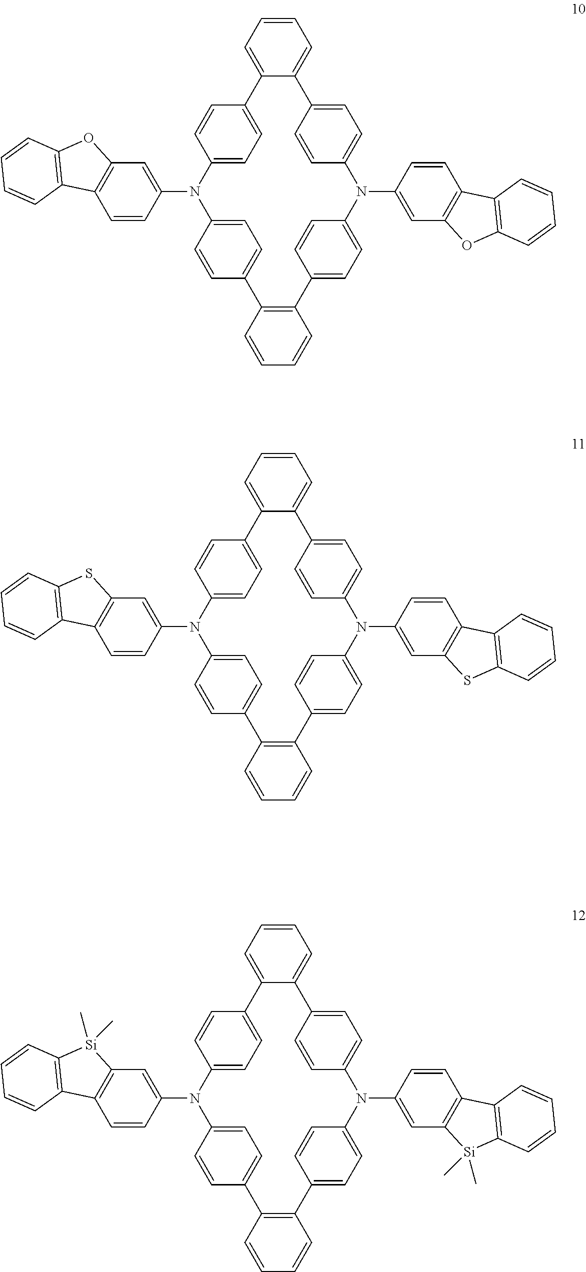 [SMILES: c1ccc2c(c1)-c1ccc(cc1)N(c1ccc3c(c1)oc1ccccc13)c1ccc(cc1)-c1ccccc1-c1ccc(cc1)N(c1ccc3c(c1)oc1ccccc13)c1ccc-2cc1 ; c1ccc2c(c1)-c1ccc(cc1)N(c1ccc3c(c1)sc1ccccc13)c1ccc(cc1)-c1ccccc1-c1ccc(cc1)N(c1ccc3c(c1)sc1ccccc13)c1ccc-2cc1 ; C[Si]1(C)c2ccccc2-c2ccc(N3c4ccc(cc4)-c4ccccc4-c4ccc(cc4)N(c4ccc5c(c4)[Si](C)(C)c4ccccc4-5)c4ccc(cc4)-c4ccccc4-c4ccc3cc4)cc21]

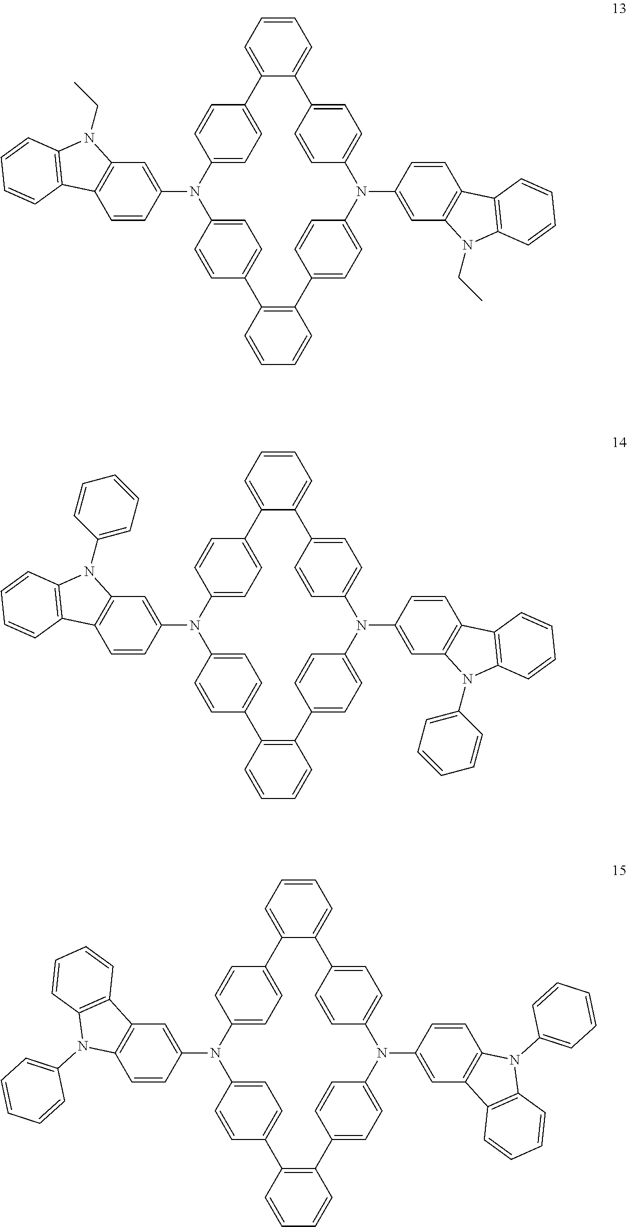 [SMILES: CCn1c2ccccc2c2ccc(N3c4ccc(cc4)-c4ccccc4-c4ccc(cc4)N(c4ccc5c6ccccc6n(CC)c5c4)c4ccc(cc4)-c4ccccc4-c4ccc3cc4)cc21 ; c1ccc(-n2c3ccccc3c3ccc(N4c5ccc(cc5)-c5ccccc5-c5ccc(cc5)N(c5ccc6c7ccccc7n(-c7ccccc7)c6c5)c5ccc(cc5)-c5ccccc5-c5ccc4cc5)cc32)cc1 ; c1ccc(-n2c3ccccc3c3cc(N4c5ccc(cc5)-c5ccccc5-c5ccc(cc5)N(c5ccc6c(c5)c5ccccc5n6-c5ccccc5)c5ccc(cc5)-c5ccccc5-c5ccc4cc5)ccc32)cc1]

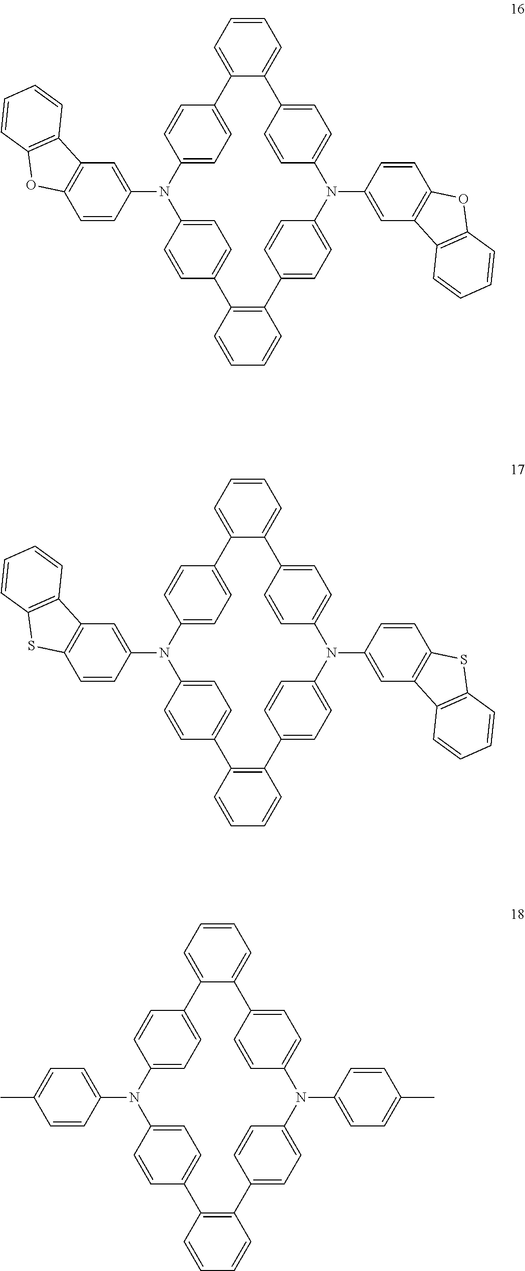

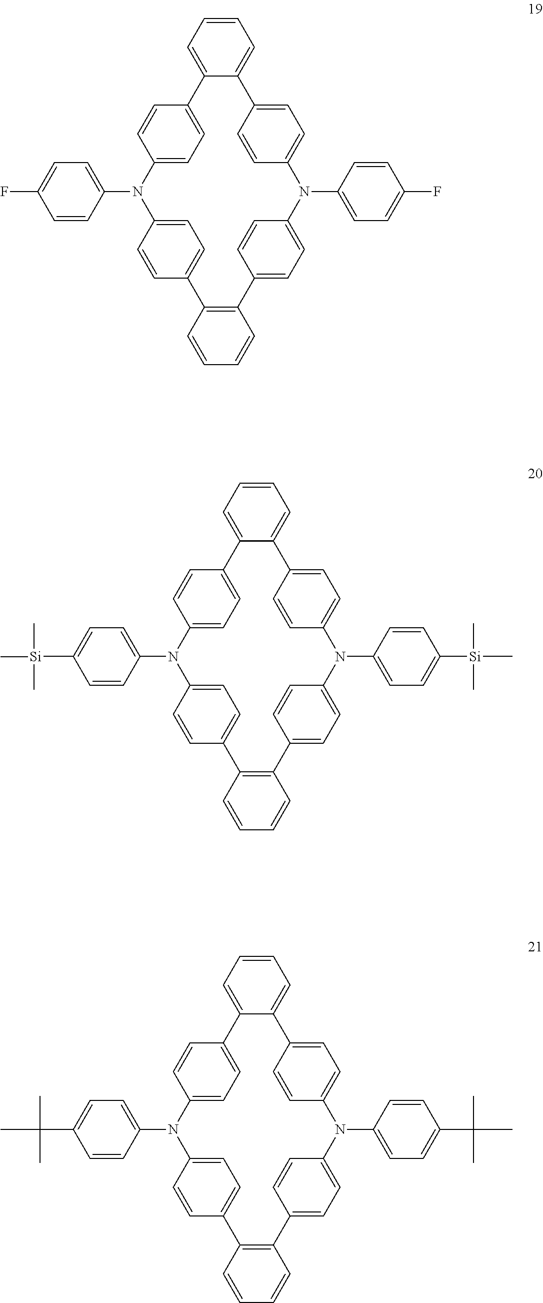

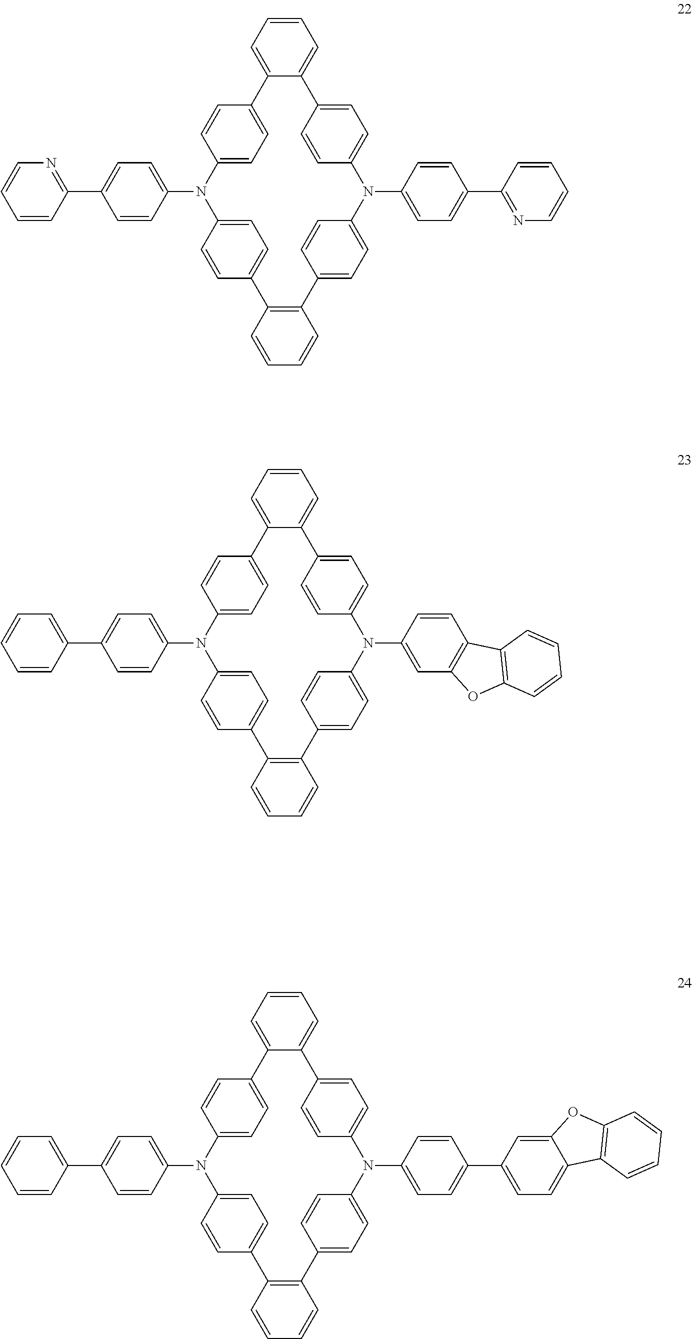 [SMILES: c1ccc(-c2ccc(N3c4ccc(cc4)-c4ccccc4-c4ccc(cc4)N(c4ccc(-c5ccccn5)cc4)c4ccc(cc4)-c4ccccc4-c4ccc3cc4)cc2)nc1 ; c1ccc(-c2ccc(N3c4ccc(cc4)-c4ccccc4-c4ccc(cc4)N(c4ccc5c(c4)oc4ccccc45)c4ccc(cc4)-c4ccccc4-c4ccc3cc4)cc2)cc1 ; c1ccc(-c2ccc(N3c4ccc(cc4)-c4ccccc4-c4ccc(cc4)N(c4ccc(-c5ccc6c(c5)oc5ccccc56)cc4)c4ccc(cc4)-c4ccccc4-c4ccc3cc4)cc2)cc1]

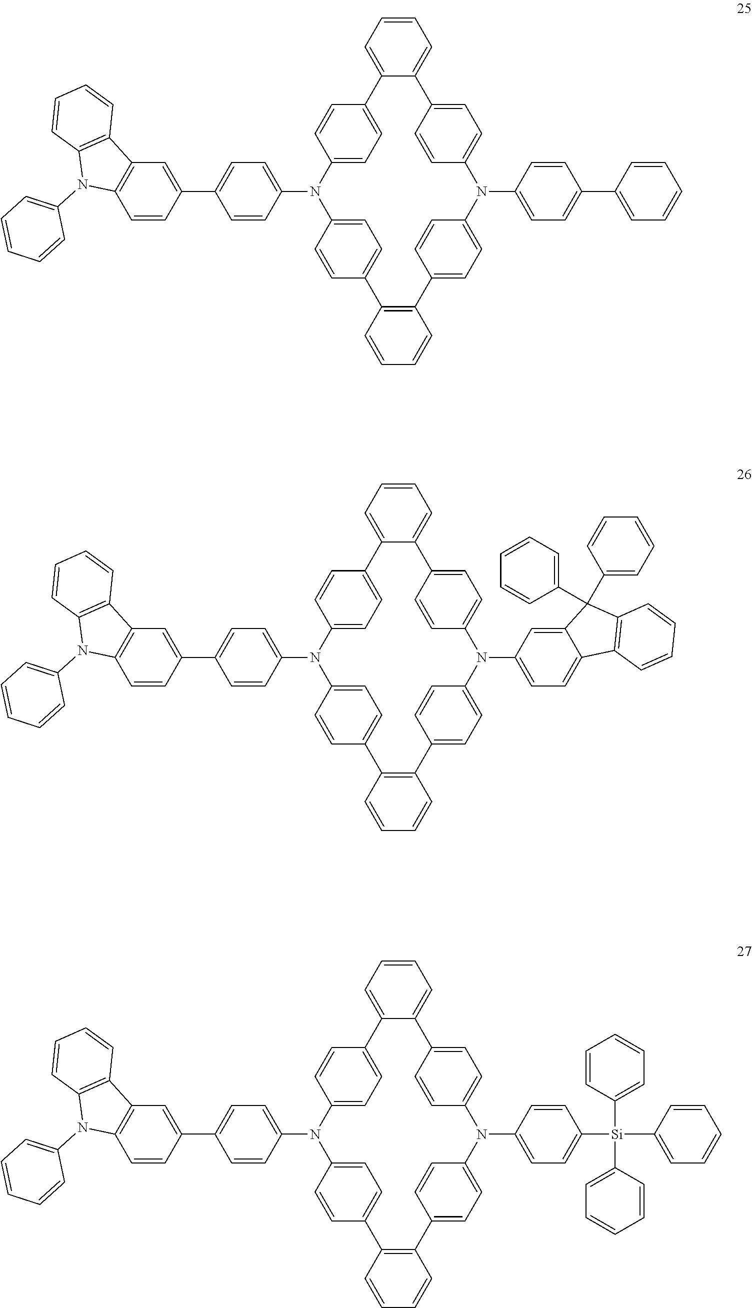 [SMILES: c1ccc(-c2ccc(N3c4ccc(cc4)-c4ccccc4-c4ccc(cc4)N(c4ccc(-c5ccc6c(c5)c5ccccc5n6-c5ccccc5)cc4)c4ccc(cc4)-c4ccccc4-c4ccc3cc4)cc2)cc1 ; c1ccc(-n2c3ccccc3c3cc(-c4ccc(N5c6ccc(cc6)-c6ccccc6-c6ccc(cc6)N(c6ccc7c(c6)C(c6ccccc6)(c6ccccc6)c6ccccc6-7)c6ccc(cc6)-c6ccccc6-c6ccc5cc6)cc4)ccc32)cc1 ; c1ccc(-n2c3ccccc3c3cc(-c4ccc(N5c6ccc(cc6)-c6ccccc6-c6ccc(cc6)N(c6ccc([Si](c7ccccc7)(c7ccccc7)c7ccccc7)cc6)c6ccc(cc6)-c6ccccc6-c6ccc5cc6)cc4)ccc32)cc1]

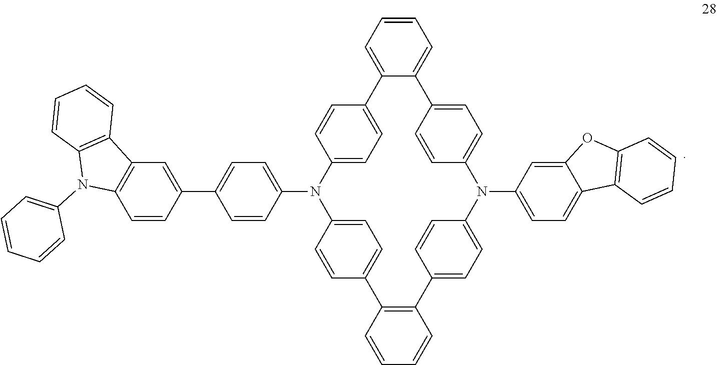 [SMILES: c1ccc(-n2c3ccccc3c3cc(-c4ccc(N5c6ccc(cc6)-c6ccccc6-c6ccc(cc6)N(c6ccc7c(c6)oc6ccccc67)c6ccc(cc6)-c6ccccc6-c6ccc5cc6)cc4)ccc32)cc1]

5. An organic electroluminescent (EL) device comprising:
an anode,
a cathode, and
an emission layer between the anode and the cathode,
wherein the emission layer comprises a material for an organic EL device represented by Formula (1): 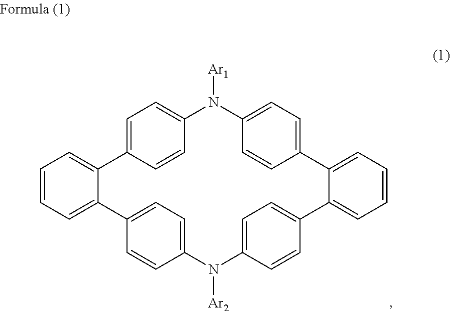
wherein, in Formula (1), Ar₁ and Ar₂ are each independently selected from a substituted or unsubstituted aryl group and a substituted or unsubstituted heteroaryl group.

6. The organic EL device of claim 5, wherein the material represented by Formula (1) comprises at least one selected from Compounds 1 to 28: 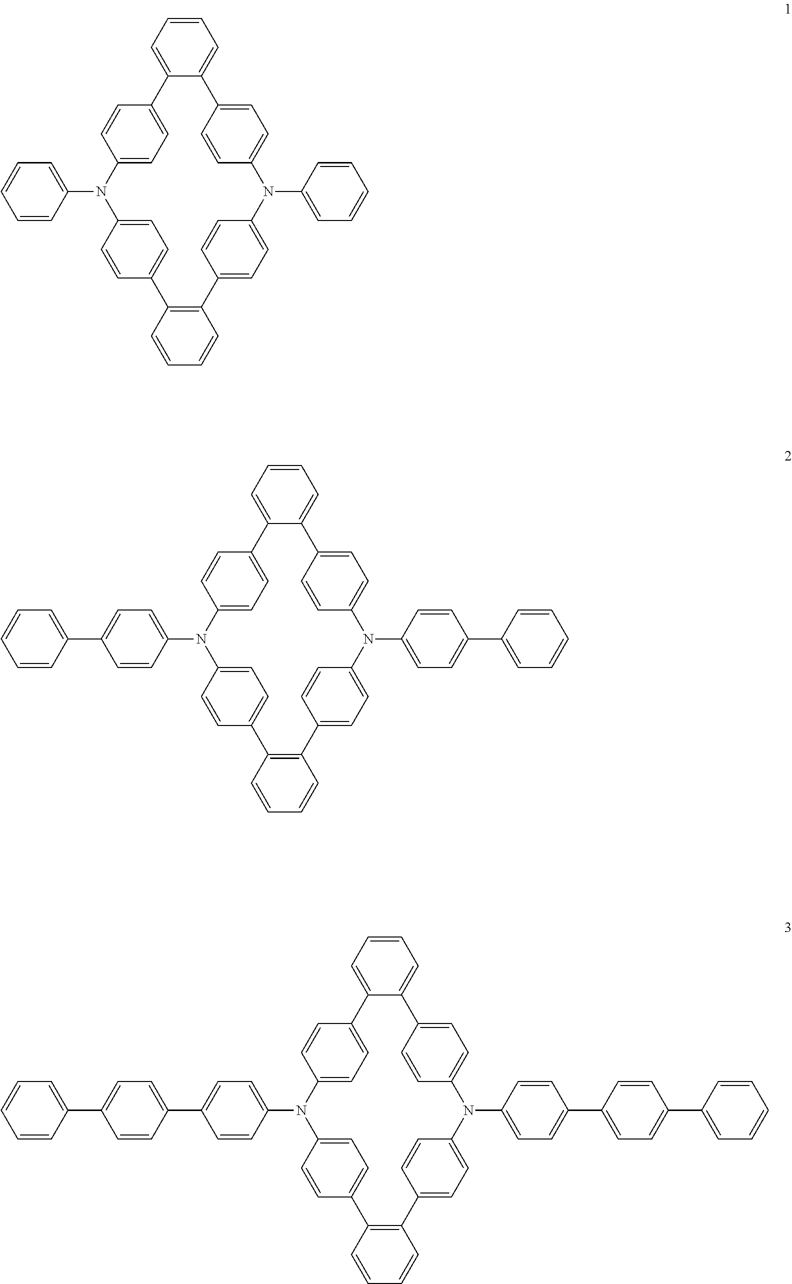 [SMILES: c1ccc(N2c3ccc(cc3)-c3ccccc3-c3ccc(cc3)N(c3ccccc3)c3ccc(cc3)-c3ccccc3-c3ccc2cc3)cc1 ; c1ccc(-c2ccc(N3c4ccc(cc4)-c4ccccc4-c4ccc(cc4)N(c4ccc(-c5ccccc5)cc4)c4ccc(cc4)-c4ccccc4-c4ccc3cc4)cc2)cc1 ; c1ccc(-c2ccc(-c3ccc(N4c5ccc(cc5)-c5ccccc5-c5ccc(cc5)N(c5ccc(-c6ccc(-c7ccccc7)cc6)cc5)c5ccc(cc5)-c5ccccc5-c5ccc4cc5)cc3)cc2)cc1] 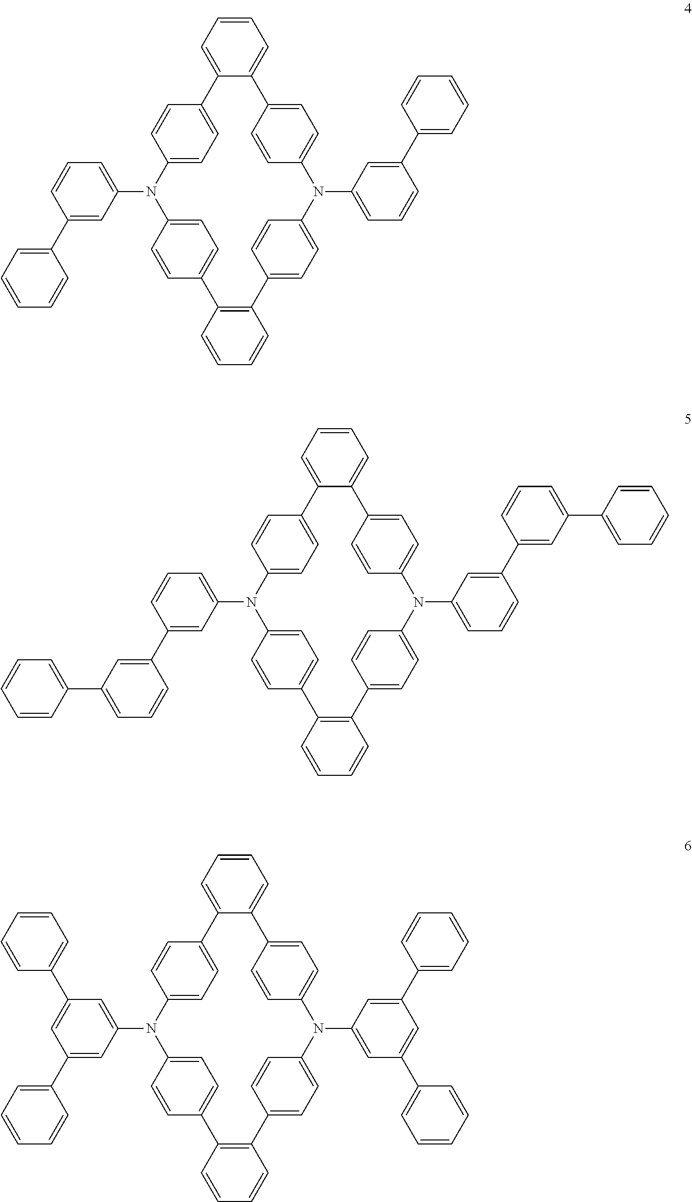 [SMILES: c1ccc(-c2cccc(N3c4ccc(cc4)-c4ccccc4-c4ccc(cc4)N(c4cccc(-c5ccccc5)c4)c4ccc(cc4)-c4ccccc4-c4ccc3cc4)c2)cc1 ; c1ccc(-c2cccc(-c3cccc(N4c5ccc(cc5)-c5ccccc5-c5ccc(cc5)N(c5cccc(-c6cccc(-c7ccccc7)c6)c5)c5ccc(cc5)-c5ccccc5-c5ccc4cc5)c3)c2)cc1 ; c1ccc(-c2cc(-c3ccccc3)cc(N3c4ccc(cc4)-c4ccccc4-c4ccc(cc4)N(c4cc(-c5ccccc5)cc(-c5ccccc5)c4)c4ccc(cc4)-c4ccccc4-c4ccc3cc4)c2)cc1] 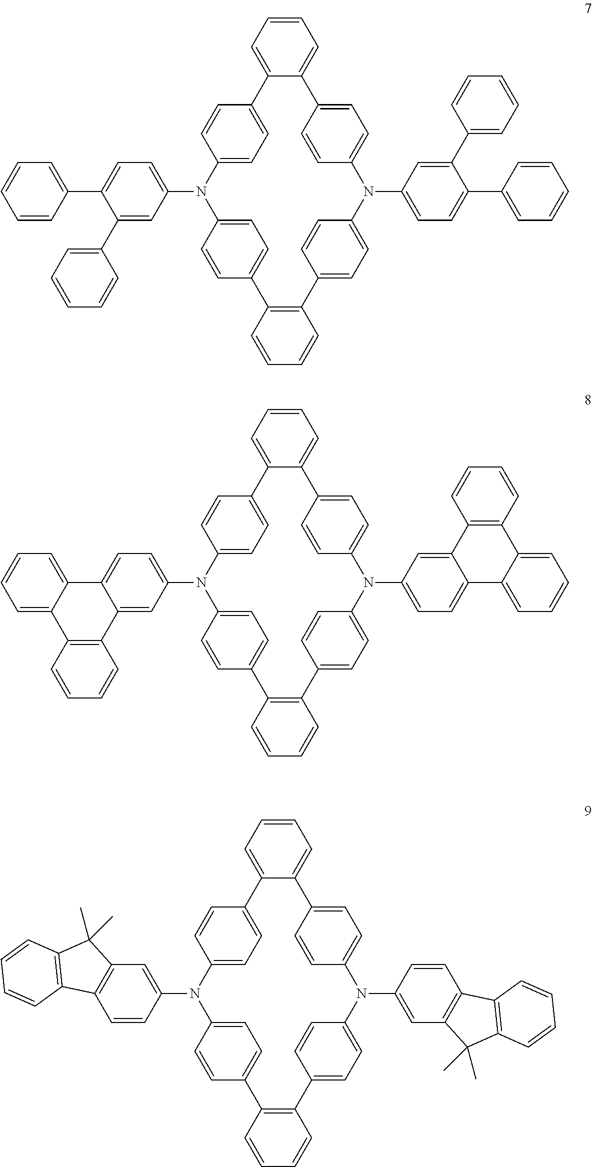 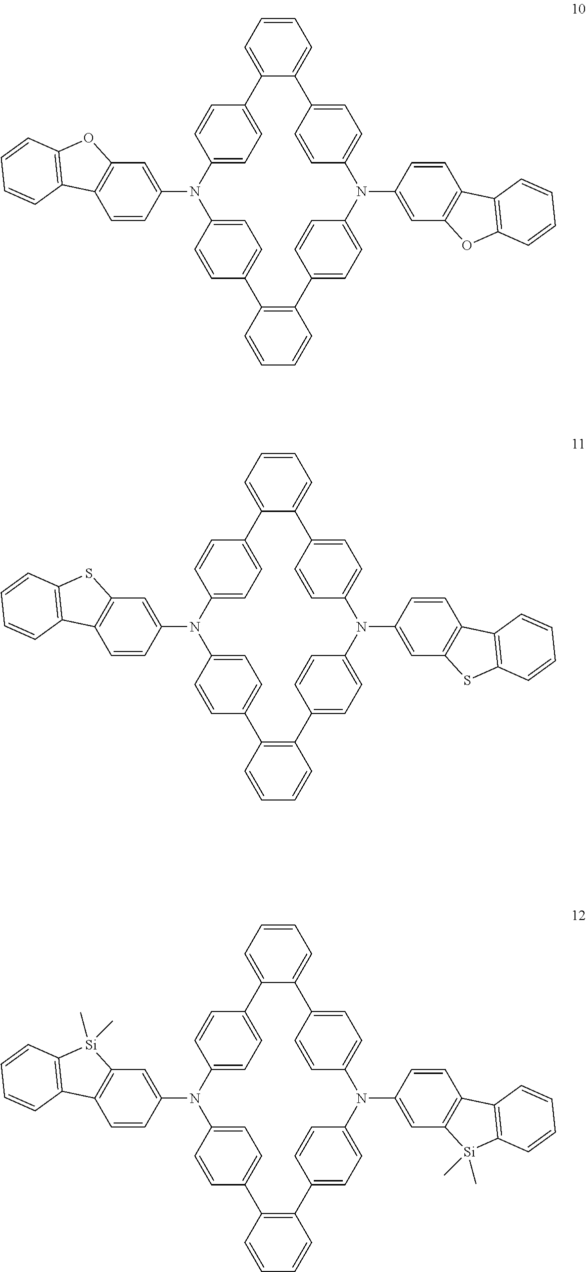 [SMILES: c1ccc2c(c1)-c1ccc(cc1)N(c1ccc3c(c1)oc1ccccc13)c1ccc(cc1)-c1ccccc1-c1ccc(cc1)N(c1ccc3c(c1)oc1ccccc13)c1ccc-2cc1 ; c1ccc2c(c1)-c1ccc(cc1)N(c1ccc3c(c1)sc1ccccc13)c1ccc(cc1)-c1ccccc1-c1ccc(cc1)N(c1ccc3c(c1)sc1ccccc13)c1ccc-2cc1 ; C[Si]1(C)c2ccccc2-c2ccc(N3c4ccc(cc4)-c4ccccc4-c4ccc(cc4)N(c4ccc5c(c4)[Si](C)(C)c4ccccc4-5)c4ccc(cc4)-c4ccccc4-c4ccc3cc4)cc21] 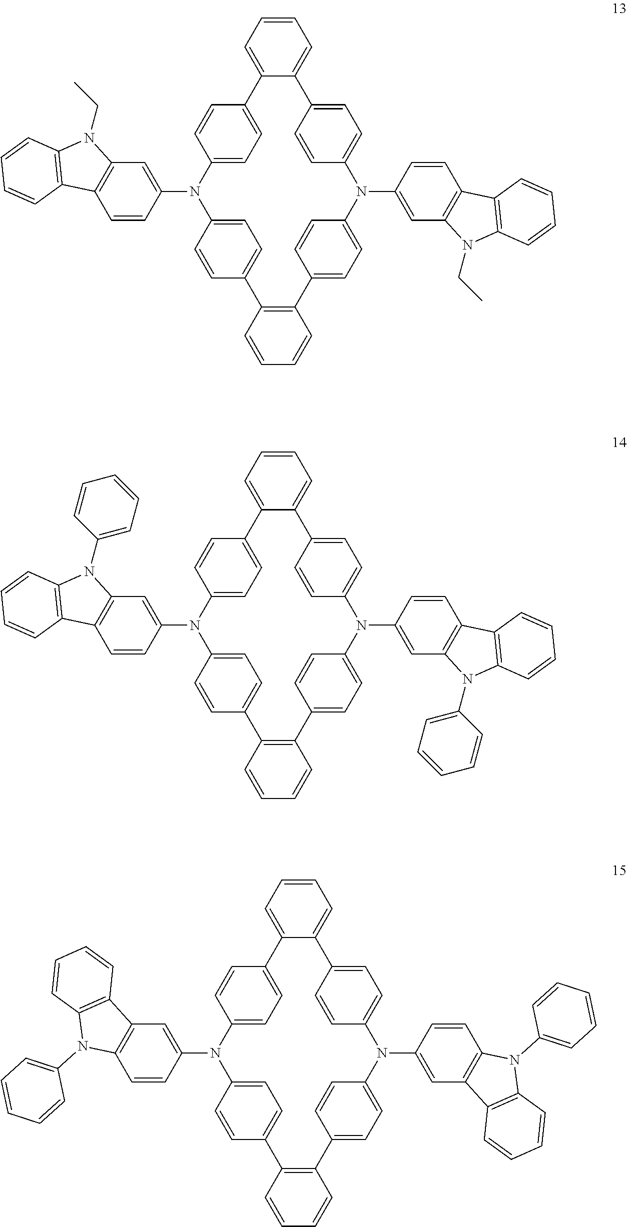 [SMILES: CCn1c2ccccc2c2ccc(N3c4ccc(cc4)-c4ccccc4-c4ccc(cc4)N(c4ccc5c6ccccc6n(CC)c5c4)c4ccc(cc4)-c4ccccc4-c4ccc3cc4)cc21 ; c1ccc(-n2c3ccccc3c3ccc(N4c5ccc(cc5)-c5ccccc5-c5ccc(cc5)N(c5ccc6c7ccccc7n(-c7ccccc7)c6c5)c5ccc(cc5)-c5ccccc5-c5ccc4cc5)cc32)cc1 ; c1ccc(-n2c3ccccc3c3cc(N4c5ccc(cc5)-c5ccccc5-c5ccc(cc5)N(c5ccc6c(c5)c5ccccc5n6-c5ccccc5)c5ccc(cc5)-c5ccccc5-c5ccc4cc5)ccc32)cc1] 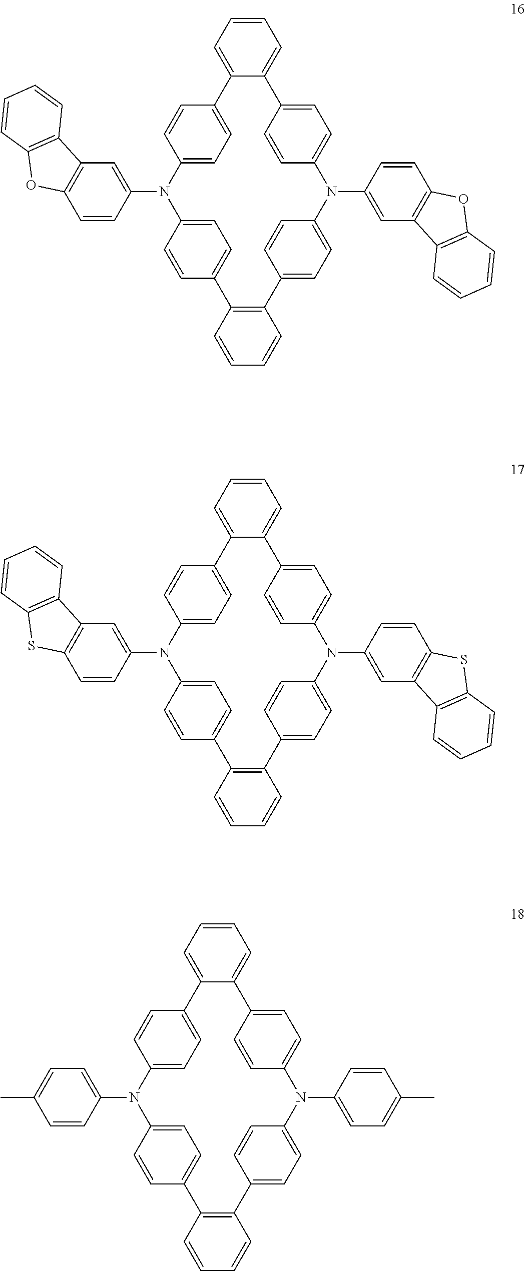 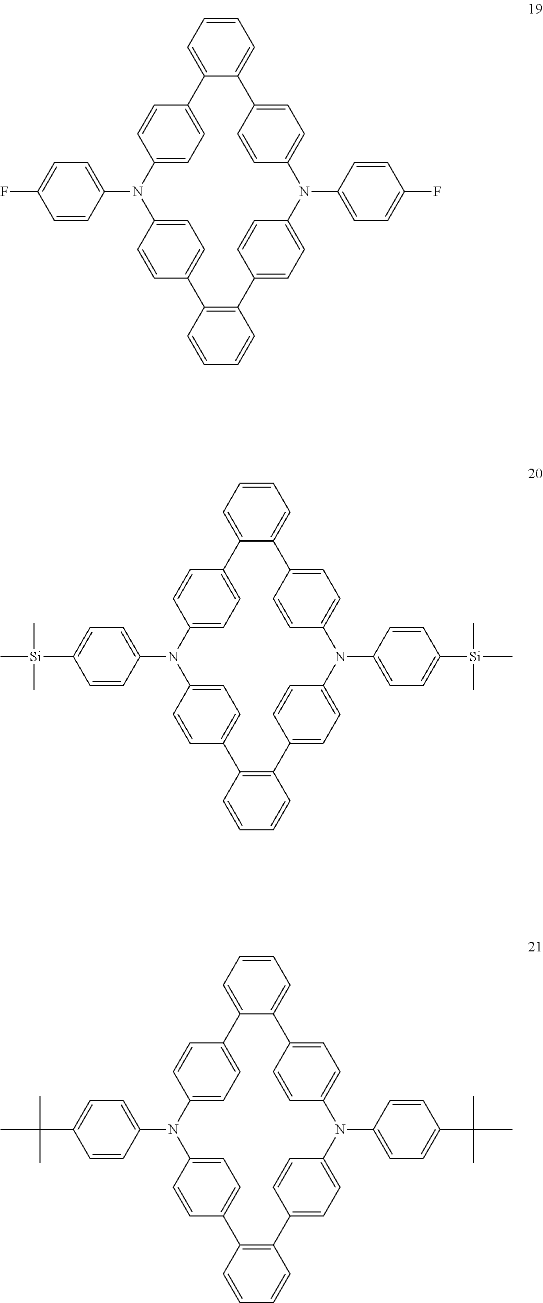 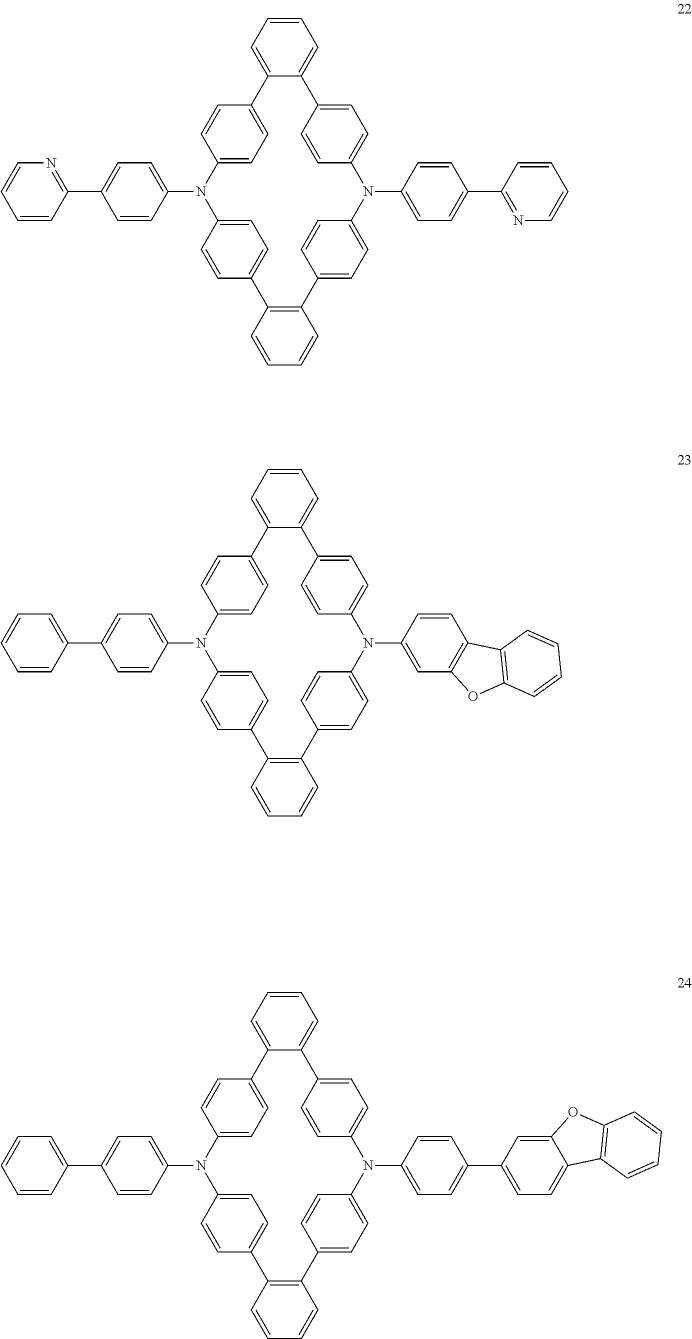 [SMILES: c1ccc(-c2ccc(N3c4ccc(cc4)-c4ccccc4-c4ccc(cc4)N(c4ccc(-c5ccccn5)cc4)c4ccc(cc4)-c4ccccc4-c4ccc3cc4)cc2)nc1 ; c1ccc(-c2ccc(N3c4ccc(cc4)-c4ccccc4-c4ccc(cc4)N(c4ccc5c(c4)oc4ccccc45)c4ccc(cc4)-c4ccccc4-c4ccc3cc4)cc2)cc1 ; c1ccc(-c2ccc(N3c4ccc(cc4)-c4ccccc4-c4ccc(cc4)N(c4ccc(-c5ccc6c(c5)oc5ccccc56)cc4)c4ccc(cc4)-c4ccccc4-c4ccc3cc4)cc2)cc1] 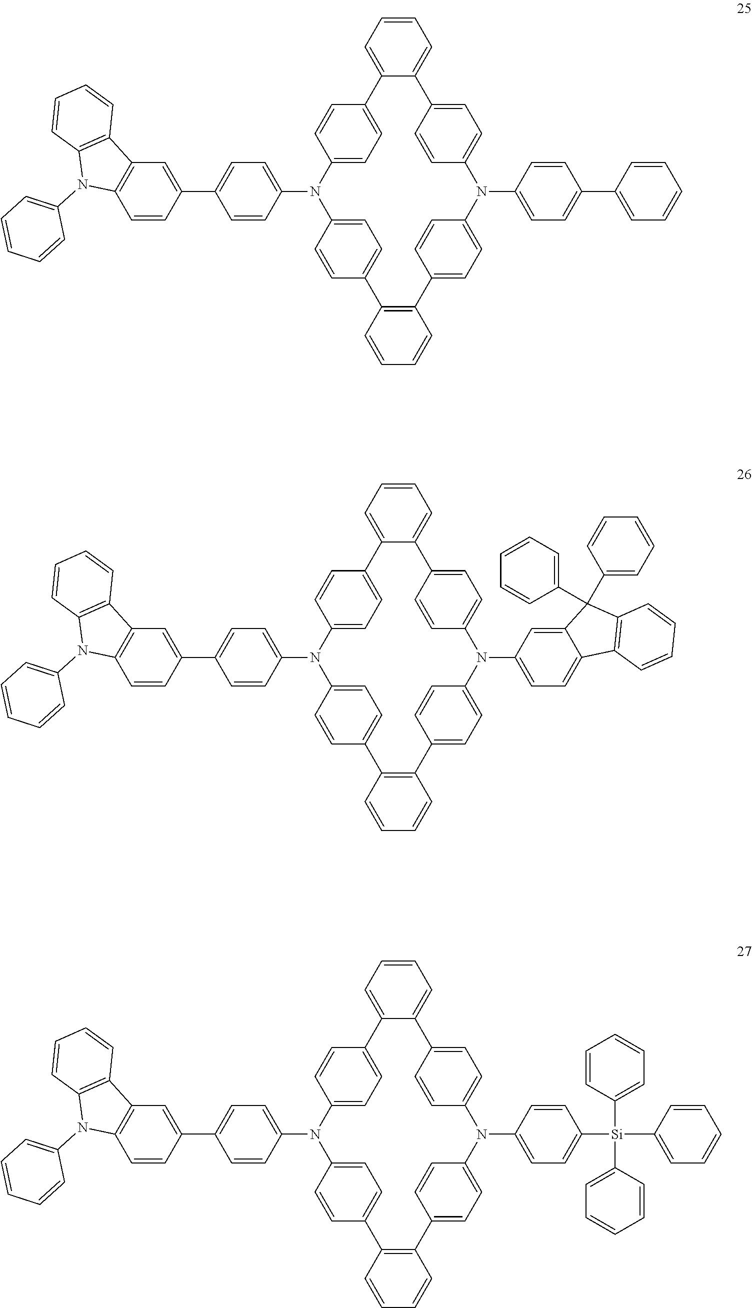 [SMILES: c1ccc(-c2ccc(N3c4ccc(cc4)-c4ccccc4-c4ccc(cc4)N(c4ccc(-c5ccc6c(c5)c5ccccc5n6-c5ccccc5)cc4)c4ccc(cc4)-c4ccccc4-c4ccc3cc4)cc2)cc1 ; c1ccc(-n2c3ccccc3c3cc(-c4ccc(N5c6ccc(cc6)-c6ccccc6-c6ccc(cc6)N(c6ccc7c(c6)C(c6ccccc6)(c6ccccc6)c6ccccc6-7)c6ccc(cc6)-c6ccccc6-c6ccc5cc6)cc4)ccc32)cc1 ; c1ccc(-n2c3ccccc3c3cc(-c4ccc(N5c6ccc(cc6)-c6ccccc6-c6ccc(cc6)N(c6ccc([Si](c7ccccc7)(c7ccccc7)c7ccccc7)cc6)c6ccc(cc6)-c6ccccc6-c6ccc5cc6)cc4)ccc32)cc1] 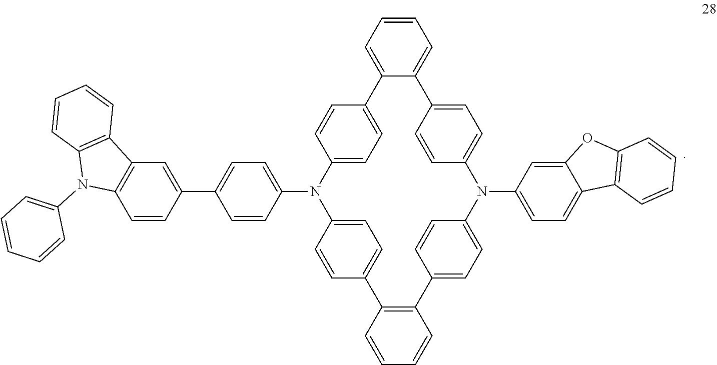 [SMILES: c1ccc(-n2c3ccccc3c3cc(-c4ccc(N5c6ccc(cc6)-c6ccccc6-c6ccc(cc6)N(c6ccc7c(c6)oc6ccccc67)c6ccc(cc6)-c6ccccc6-c6ccc5cc6)cc4)ccc32)cc1]
